(19) United States
(12) Patent Application Publication
(10) Pub. No.: US 2026/0048248 A1
(43) Pub. Date: Feb. 19, 2026

(54) WOUND DRESSINGS

(71) Applicant: ConvaTec Limited, Flintshire (GB)

(72) Inventors: Nicholas David Boote, Flintshire (GB); Hugh Semple Munro, Flintshire (GB)

(21) Appl. No.: 19/183,894

(22) Filed: Apr. 20, 2025

Related U.S. Application Data
(63) Continuation of application No. PCT/GB2023/052737, filed on Oct. 20, 2023.

(30) Foreign Application Priority Data
Oct. 20, 2022 (GB) 2215521.2

(51) Int. Cl. A61M 35/00; A61L 26/00; B65D 65/38

(52) U.S. Cl. CPC A61M 35/30; A61L 26/0004; A61L 26/0066; B65D 65/38; A61M 2202/0275; A61M 2205/0205; A61M 2205/0238; A61M 2209/06

(57) ABSTRACT

A wound dressing for treating a wound, the wound dressing comprising a nitric oxide generating layer for generating nitric oxide by the acidification of a nitrite salt, wherein the nitric oxide generating layer includes a solid powder nitrite salt component and a solid proton source component, methods and uses of such a wound dressing for treating a wound.

The present invention relates to wound dressings for treating a wound, the wound dressing comprising a nitric oxide generating layer for generating nitric oxide by the acidification of a nitrite salt, wherein the nitric oxide generating layer includes both a solid powder nitrite salt component and a solid proton source component.

### BACKGROUND

Nitric oxide (NO) and nitric oxide precursors have been extensively studied as potential pharmaceutical agents.

However, there remain substantial problems in connection with the efficient generation and delivery of nitric oxide, other oxides of nitrogen and precursors thereof to organisms and cells for treatment. A widely adopted system for the generation of nitric oxide relies on the acidification of nitrite salts using a proton source such as an acid to produce initially nitrous acid (HNO₂), which nitrous acid then readily decomposes to nitric oxide and nitrate with hydrogen ions and water. The decomposition can be represented by the following balanced equation (1):

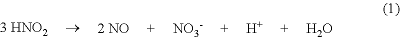

The acid and nitrite salt are typically provided as separate components at pre-determined quantities. The separate components are kept apart until the point of use to minimize reaction before the point of need. These two reactants are thus provided in a two-part system involving a part containing the nitrite salt and a separate part containing the acid. In this way the two separate components in the two separate parts can be combined or mixed at the point of need to prevent the release of nitric oxide before required.

The properties of nitric oxide, for example, to kill or prevent the proliferation of microbes have given rise to an important utility in the treatment of wounds, skin lesions and burns. Therefore, means for generating and delivering nitric oxide have also been applied to wound dressings. However, there remains a need for the acid and nitrite to be kept separate until the point of use and so two-part wound dressing systems have been developed.

Typically, such two-part wound dressings include a first part including the nitrite salt and second part including the acid, and these two components are kept separate until the point of need. The two-part wound dressing is therefore packaged as two separate pieces and then combining of these two separate pieces is required in order to initiate reaction. The use of a two-part system has its drawbacks. For example, the combination of a two-part system at the point of need has the potential to introduce, for example, use errors or dosing inaccuracies when the two parts are combined.

In contrast, a one-part (also referred to as single-part) wound dressing provides the wound dressing in a single piece before the point of use. One-part wound dressings are simpler to use because they typically only require minimal preparation (e.g. removal of a protective film) before application to a subject. It is therefore desirable to provide a nitrite salt and an acidification source as a one-part or single part wound dressing. In addition, the provision of a single part product may reduce manufacturing complexity, cost, and packaging.

However, difficulties arise when attempting to provide the acidification of nitrite as a single part system because the reaction would initiate, and the system would lose a significant proportion of its nitric oxide during manufacturing and storage and could potentially not provide enough nitric oxide at the point of need stage.

WO 2021/198461 describes a wound dressing for generating nitric oxide. The wound dressing described requires that the nitric oxide-releasing agent and the activator (species for activating and/or facilitating release of nitric oxide from the nitric oxide-releasing agent) are present in the wound dressing as two separate and distinct layers (the nitric oxide source layer and the activator layer). In addition to the separate and distinct layers, use of a separating layer, to further prevent contact between the nitric oxide source layer and the activator layer, prior to use, is also described.

There is therefore a need for a single-part wound dressing system, which provides the species necessary for generating and delivering nitric oxide, such as a nitrite salt and acid species, without causing premature or unwanted release of NO prior to the point of need.

### SUMMARY OF THE INVENTION

The present inventors have sought to provide a simple wound dressing for delivering nitric oxide by acidification of nitrite.

At its most general, the present invention provides a wound dressing having a nitric oxide generating layer and the nitric oxide generating layer includes both a solid of a nitrite salt and a solid of a proton source. In this way, the nitrite salt and the proton source are held in close proximity (or intimately associated) to provide acidification of the nitrite when in contact with an aqueous environment, but do not substantially react until required, and therefore a single component system may be provided. Including solid components of both the nitrite salt and the acid source may avoid the inclusion of a source of moisture (such as a solution or an aqueous-based gel). In this way, the reactants have reduced exposure to moisture to minimise reaction before a reaction is needed.

In a first aspect, the present invention provides a wound dressing for treating a wound, the wound dressing comprising a nitric oxide generating layer for generating nitric oxide by the acidification of a nitrite salt, wherein the nitric oxide generating layer includes a solid powder nitrite salt component and a solid proton source component.

The nitric oxide generating layer may include a dry wound dressing substrate. The solid powder nitrite salt is typically admixed with the dry wound dressing substrate. In particular embodiments, the dry proton source component includes a solid powder proton source component, and the solid powder proton source component is admixed with the dry wound dressing substrate. In other embodiments, at least part of the dry proton source component forms part of the dry wound dressing substrate. For example, the dry wound dressing substrate may include proton source fibres. In certain embodiments, the dry proton source component includes a solid powder proton source component and further part of the dry proton source component forms part of the dry wound dressing substrate.

The dry wound dressing substrate may be made up of woven or non-woven fibres.

All of the components of the nitric oxide generating layer may be dry components. The water content of the nitric oxide generating layer may be 10% or less, 5% or less, 2% or less or 1% or less based on the weight of the nitric oxide generating layer.

The wound dressing may be a one-part wound dressing. In other words, the wound dressing may be provided as a single piece prior to the point of need.

The wound dressing may include one or more further layers in addition to the nitric oxide generating layer. In other words, the wound dressing may be a multi-layer wound dressing. The nitric oxide generating layer may be composited with other layers and/or materials to make the wound dressing.

The solid powder nitrite salt component and the solid powder proton source component may be provided by:
a. A blend of one or more individual particles containing a nitrite salt and one or more individual particles containing a proton source;
b. one or more individual particles that each contain a nitrite salt and a proton source;
c. an agglomeration of particles, wherein the agglomeration of particles includes one or more individual particles containing a nitrite salt, one or more individual particles containing a proton source and optionally a binding agent;
d. an agglomeration of particles, wherein the agglomeration of particles includes one or more individual particles that each contain a nitrite salt and a proton source and optionally a binding agent; or
e. combinations thereof.

The individual particles or agglomeration of particles may be blended with or coated with an excipient for affecting the rate of water ingress into particles and/or an excipient for affecting the kinetics of the formation of nitric oxide from the particles.

The excipient for affecting the rate of water ingress into particles may be a polyol or a hydrophobic material, such as one or more phospholipids (e.g. dipalmitoylphosphatidylcholine, DPPC) magnesium stearate or colloidal silica, and/or the excipient for affecting the rate of water ingress into particles may be a nitric oxide or nitric oxide precursor sequestering material, such as thiols, alcohols, amines or amides.

The particles containing a nitrite salt and a proton source may be formed by spray-drying a mixture containing a nitrite salt solution and a proton source solution.

The blend of one or more individual particles containing a nitrite salt and one or more individual particles containing a proton source may be formed by either (a) micronizing a nitrite salt solid with a proton source solid; or (b) combining two solids by:
(i) spray-drying or lyophilising a solution containing a nitrite salt,
(ii) spray-drying or lyophilising a solution containing a proton source, and
(iii) blending the solids generated in steps (i) and (ii).

The proton source may comprise an acid precursor, such as an ester or a photoacid.

The wound dressing may include one or more further dry layers adjacent to nitric oxide generating layer. The water content of any layer adjacent to the nitric oxide generating layer may be 10% or less, 5% or less, 2% or less or 1% or less based on the weight of the layer adjacent to the nitric oxide generating layer.

The wound dressing may further include an anti-microbial agent.

In a second aspect, the present invention provides a packaged wound dressing comprising a wound dressing as described herein within a low moisture permeability packaging. The low moisture permeability packaging may include one or more low moisture permeability materials (e.g. aluminium foil) in the walls of the packaging and/or may be hermetically sealed.

The packaging atmosphere within the packaged wound dressing may have a low moisture content at initial packaging and/or the package includes pack inserts that sequester moisture.

In a third aspect, the present invention provides a method of treating a wound, the method comprising applying a wound dressing as described herein to wound of a subject. The wound dressing may be a one-part wound dressing. In other words, the wound dressing may be provided as a single piece prior to the point of need.

In a fourth aspect, the present invention provides a combination of a solid powder nitrite salt component and a solid proton source component in a wound dressing as described herein for use in treating a wound in a subject. The wound dressing may be a one-part wound dressing. In other words, the wound dressing may be provided as a single piece prior to the point of need.

The optional or particular features of one aspect of the invention as described herein apply equally to the other aspects of the present invention in so far as that feature is compatible with the aspect. In particular, the optional or particular features of the wound dressing apply equally to the packaged wound dressing, method of treating a wound and combination for use in treating a wound in so far as these features are compatible with those parts.

### DETAILED DESCRIPTION

The present invention will now be described in more detail. The examples and the following figures provide exemplification of the invention.

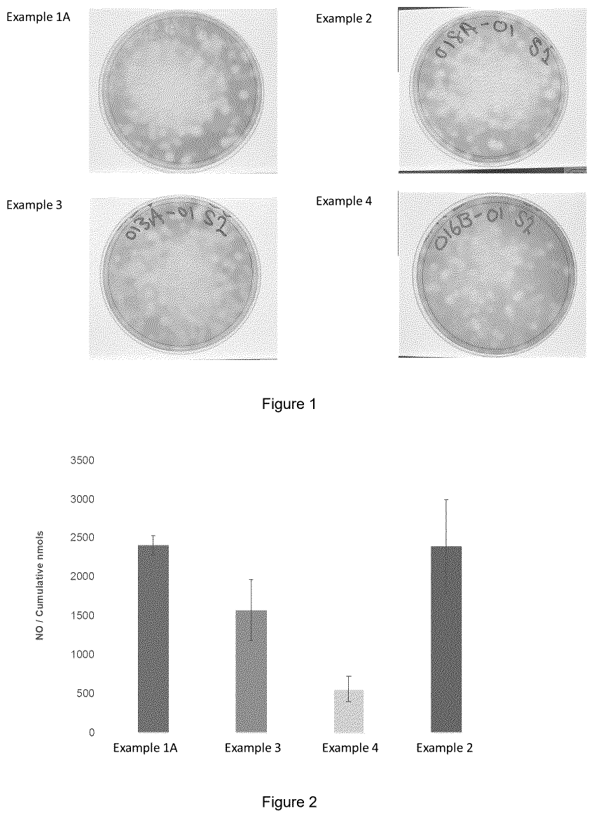

FIG. 1 shows the deposition pattern of powder of Examples 1A, 2, 3 and 4 on agarose with Hanks' balanced salt solution and a pH indicator (phenol red).

FIG. 2 shows the cumulative NO generation for Examples 1A, 2, 3 and 4.

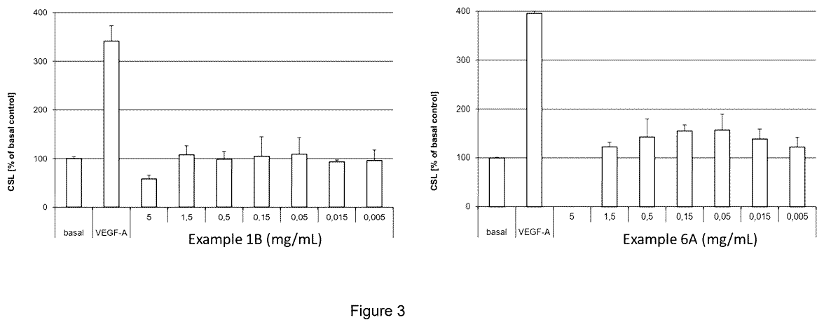

FIG. 3 shows the sprouting intensity of HUVEC spheroids treated with the Examples 1B and 6A quantitated by an image analysis system to determine the cumulative sprout length per spheroid (CSL) relative to the basal control.

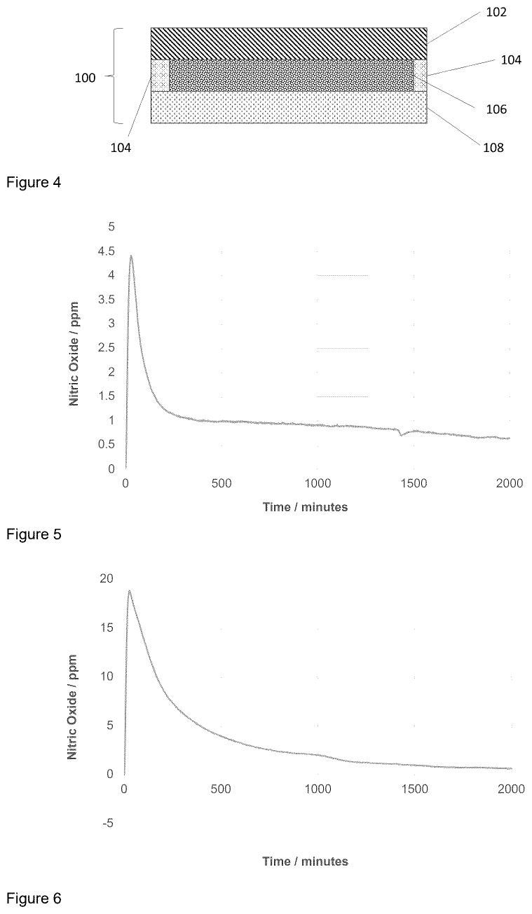

FIG. 4 shows a schematic of a wound dressing of the present invention.

FIG. 5 shows an NO release profile for a wound dressing of the present invention over 2000 minutes.

FIG. 6 shows an NO release profile for another wound dressing of the present invention over 2000 minutes.

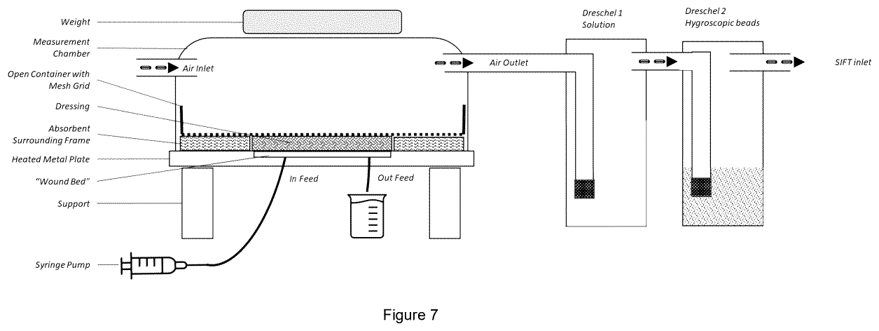

FIG. 7 shows a schematic of an apparatus used to measure and analyse the evolved gaseous nitric oxide by Selected-Ion Flow Tube Mass Spectrometry (SIFT-MS).

The reaction between one or more nitrite salt and a proton source to generate nitric oxide, optionally other oxides of nitrogen and/or optionally precursors thereof is referred to herein as the “NOx generating reaction” or the “reaction to generate NOx” or like wording, and “NOx” is used to refer to the products of the acidification of nitrite, particularly nitric oxide, other oxides of nitrogen and precursors thereof both individually and collectively in any combination. It will be understood that each component of the generated NOx can be evolved as a gas, or can pass into solution in the reaction mixture, or can initially pass into solution and subsequently be evolved as a gas, or any combination thereof.

The term “about” is used herein to denote that the numerical value is not strictly limiting and the skilled person will understand that the value may extend above or below (as appropriate) the exact value in line with the skilled person's understanding of the value. The term “about” may signify a value that is up to ±10% of the value.

Particle size as described herein refers to the volume mean diameter (VMD), unless stated otherwise.

The use of the terms “one-part”, “single-part”, “two-part” and “multi-part” as used herein refers to the number of pieces of the wound dressing prior to the point of need (e.g. application to a subject). For example, a one-part wound dressing is provided as a single piece prior to the point of need. The one-part wound dressing is typically applied to the subject as a single piece. In contrast, two-part and multi-part wound dressings are provided in two or multiple pieces, respectively, prior to the point of need and typically combined into a single piece wound dressing just before applying to the subject. It should also be noted that one-part wound dressings as described herein may be formed from the nitric oxide generating layer and one or more other layers or components, such as a backing layer.

### Wound Dressings

A “wound dressing” as used herein is a material that is intended to be applied to an exterior surface (e.g. skin or fur) of a subject (e.g. a human or animal) to cover, protect and/or treat a lesion on the skin of the subject. A wound dressing is suitable for use in relation to any breakage or interruption in the skin barrier which can be caused by, for example, ulcers, surgery, burns, cuts, lesions, wounds, lacerations, trauma and/or abrasions.

### Nitric Oxide Generating Layer

The wound dressings of the present invention comprise a nitric oxide generating layer for generating nitric oxide by the acidification of a nitrite salt, wherein the nitric oxide generating layer includes a solid powder nitrite salt component and a solid proton source component.

In this way, the nitrite salt and proton source may be in close proximity to sufficiently react when exposed to an aqueous environment. The nitrite salt and proton source are present in a single layer of the wound dressing. In this way, these components do not need to be combined (e.g. as part of a two-component system) at the point of use. In addition, as the nitrite salt and proton source are in solid form, the generation of nitric oxide before use is reduced as water content may be minimised.

Without wishing to be bound by theory, the inventors of the present invention found that it was possible to provide nitrite salt and proton source together in a single layer of a wound dressing, when the species were provided as a dry solid powder composition. In this form the nitrite salt and proton source are unable to react with one another and therefore do not generate NOx. However, upon exposure to a moist environment, for example, exposure to wound exudate, the nitrite salt and proton source are able to react and generate NOx. Therefore, the wound dressing of the present invention enables the nitrite salt and proton source to reside in close proximity to one another, without the need for separation either in separate layers or by a barrier layer in order to prevent the generation of NOx prior to the point of use (i.e., enables the nitrite salt and proton source to be provided on the same layer in a wound dressing).

Typically, all of the components of the nitric oxide generating layer are dry components. In this way, the reaction of the nitrite salt and acid components is minimised prior to the point of use. The water content of the nitric oxide generating layer may be 10% or less, 5% or less, 2% or less or 1% or less based on the weight of the nitric oxide generating layer. In this way, reaction between the nitrite salt and proton source reactants is minimised prior to use. The water content may be measured by standard laboratory methods, such as the weighing the sample, removing the moisture (e.g. by drying in an oven at over 100° C.) and then weighing the sample again.

### Dry Wound Dressing Substrate

The nitric oxide generating layer may include a dry wound dressing substrate. The solid powder nitrite salt is typically admixed with the dry wound dressing substrate. In particular embodiments, the dry proton source component includes a solid powder proton source component, and the solid powder proton source component is admixed with the dry wound dressing substrate. In other embodiments, at least part of the dry proton source component forms part of the dry wound dressing substrate. For example, the dry wound dressing substrate may include proton source fibres. In certain embodiments, the dry proton source component includes a solid powder proton source component and further part of the dry proton source component forms part of the dry wound dressing substrate.

Dry wound dressing substrates are known per se. The dry wound dressing may be adsorbent. The dry wound dressing substrate may be a synthetic or natural polymer species. The dry wound dressing substrate may be made up of woven or non-woven fibres or a solid foam. The dry wound dressing substrate may be made up from fibres of cotton, rayon, polyester (such as PLGA) and/or gelling fibres, such as alginate (salts of alginic acid) and carboxymethylcellulose and salts thereof. Additionally or alternatively, the dry wound dressing substrate may be a solid foam of a hydrophilic material (e.g. silicone) and/or alginate.

Particles or agglomeration of particles of the solid powder nitrite salt component and/or the solid powder proton source component may be incorporated or encapsulated into the substrate. In this way, the solid powder composition may be held within the material by the substrate until exposure with moisture or an aqueous environment. Particles of the solid powder composition may be exposed or partially exposed on the surface of the substrate or may be wholly encapsulated in the substrate.

The material may be a fibrous material comprising fibres of the substrate and particles or agglomeration of particles of the solid powder nitrite salt component and/or the solid powder proton source component may be incorporated or encapsulated into the fibrous material. Particles or agglomeration of particles of the solid powder nitrite salt component and/or the solid powder proton source component may be exposed or partially exposed on the surface of the substrate fibres or may be wholly encapsulated in the fibrous network and fibre cross-sections.

In some examples, the dry wound dressing substrate is porous and at least some of the particles or agglomeration of particles of the solid powder nitrite salt component and/or the solid powder proton source component are in the pores of the substrate. In other words, the substrate may be porous and impregnated with particles or agglomeration of particles of the solid powder nitrite salt component and/or the solid powder proton source component. In some examples, the substrate is porous by including pores in the surface of the substrate. In other examples, the substrate may be a porous mesh of substrate elements, such as polymeric fibres, and the particles or agglomeration of particles are in voids between the substrate elements. As a particular example, the particles or agglomeration of particles of the solid powder nitrite salt component and/or the solid powder proton source component may be impregnated into voids of a polymeric fibre mesh.

The particles or agglomeration of particles of the solid powder nitrite salt component and/or the solid powder proton source component may be a suitable particle size for dispersion in gelling fibres. The particles or agglomeration of particles of the solid powder nitrite salt component and/or the solid powder proton source component may have a particle size of greater than about 5 μm. For example, the particles or agglomeration of particles of the solid powder nitrite salt component and/or the solid powder proton source component may have a particle size of greater than about 50 μm, greater than about 100 μm, greater than about 250 μm, greater than about 500 μm, greater than about 750 μm, greater than about 1000 μm.

To achieve larger particle sizes, the particles or agglomeration of particles may undergo granulation. “Granulation” refers to a process of combining particulate species to form larger particles known as granules. Granulation may occur, for example, by compressing the particles or agglomerates to provide tablets which can then be broken up into granules. The particles or agglomerates may be compressed at about 1 to about 10 MT (metric tonnes), for example, may be compressed at about 3 to about 7 MT. The particles or agglomerates may be compressed at about 3.8 MT. The particles or agglomerates may be compressed at about 6.5 MT. The tablets may be broken up into granules using a sieve, for example, a 1 mm sieve.

To promote compression, a binding agent may be added to the particles or agglomerates. Suitable binding agents may include sugars, natural binders or synthetic or semisynthetic polymer binders. Sugar species may include, for example, sucrose or liquid glucose. Natural binders may include, for example, acacia, tragacanth, gelatin, starch paste, pregelatinized starch, alginic acid or cellulose. Synthetic or semisynthetic polymer binders may include, for example, methyl cellulose, ethyl cellulose, hydroxy propyl methyl cellulose (HPMC), hydroxy propyl cellulose, sodium carboxy methyl cellulose, polyvinylpyrrolidones (PVP), polyethylene glycols (PEG), polyvinyl alcohols, polymethacrylates. The binding agent may be a copolymer of 1-vinyl-2-pyrrolidone and vinyl acetate (copovidone). The binding agent may be microcrystalline cellulose.

The binding agent may be incorporated into the composition in % w/w of about 5% w/w to about 30% w/w. For example, the binding agent may be incorporated into the composition in a % w/w of about 10% w/w to about 25% w/w.

Alternatively, the composition may be substantially free of binding agents.

Particle size may be increased by such means in order to ensure that the particles or agglomerate particles remain trapped (incorporated or encapsulated) between the fibres.

The particles or agglomeration of particles of the solid powder nitrite salt component and/or the solid powder proton source component may be incorporated into the substrate when producing the substrate. A method of incorporating or encapsulating particles or agglomeration of particles of the solid powder nitrite salt component and/or the solid powder proton source component into a substrate, the method includes the steps of (i) mixing the particles or agglomeration of particles of the solid powder nitrite salt component and/or the solid powder proton source component with a non-polar liquid containing the substrate or substrate precursor to form a liquid-particle mixture and (ii) solidifying the liquid-particle mixture to form a material incorporating or encapsulating particles or agglomeration of particles of the solid powder nitrite salt component and/or the solid powder proton source component.

The liquid-particle mixture may be solidified by spinning the mixture into fibres. Techniques known to a person of skill in the art for the spinning of the fibres may be used. For example, the liquid-particle mixture may be solidified by dry spinning, wet spinning, gel spinning or electrospinning. The liquid-particle mixture may be solidified by electrospinning. “Electrospinning” refers to a fibre production method which uses electric force to draw charged threads of polymer solutions or polymer melts to fibre diameters. The liquid-particle mixture may be solidified by gel spinning. “Gel spinning” refers to a fibre production method which relies on temperature-induced physical gelation for solidification.

Alternatively, the particles or agglomeration of particles of the solid powder nitrite salt component and/or the solid powder proton source component may be incorporated into the substrate after the substrate has formed. For example, particles or agglomeration of particles of the solid powder nitrite salt component and/or the solid powder proton source component may be impregnated into a porous substrate, such as a fibrous mesh substrate. In these examples, the substrate is already formed and the particles or agglomeration of particles of the solid powder nitrite salt component and/or the solid powder proton source component are being added to it. A particular example of methods to impregnate solid powder compositions into porous substrates include those described in EP2331309 (and other techniques available from Fibroline France).

### Solid Powder Nitrite Salt Component and Solid Proton Source Component

The wound dressings of the present invention include the solid powder nitrite salt component and the solid proton source component in a single nitric oxide generating layer. In this way, the single nitric oxide generating layer may release nitric oxide by the acidification of the nitrite salt upon exposure to an aqueous environment or to moisture in the atmosphere.

### Solid Powder Nitrite Salt Component

The solid powder nitrite component includes a nitrite salt. The choice of nitrite salt is not particularly limited. The nitrite salt may be selected from one or more alkali metal nitrite salts or alkaline metal nitrite salts. For example, the one or more nitrite salt may be selected from LiNO₂, NaNO₂, KNO₂, RbNO₂, CsNO₂, FrNO₂, AgNO₂, Be(NO₂)₂, Mg(NO₂)₂, Ca(NO₂)₂, Sr(NO₂)₂, Mn(NO₂)₂, Ba(NO₂)₂, Ra(NO₂)₂ and any mixture thereof. The nitrite salt may be NaNO₂ or KNO₂. The nitrite salt may be NaNO₂.

The nitrite salt may be a pharmaceutically acceptable grade of nitrite salt. In other words, the nitrite salt may adhere to one or more active pharmacopoeia monographs for the nitrite salt. For example, the nitrite salt may adhere to the monograph of the nitrite salt of one or more of the United States Pharmacopoeia (USP), European Pharmacopoeia or Japanese Pharmacopoeia.

In particular, the nitrite salt used may have one or more of the characteristics as provided in paragraphs to and/or Table 1 in paragraph of WO 2010/093746, the disclosure of which is incorporated herein by reference in its entirety.

### Solid Proton Source Component

The solid proton source component includes a proton source. The proton source may be any species capable of acting as a source of protons for the acidification of nitrite. The choice of proton source is not particularly limited. The proton source may be, for example, an acid.

The solid proton source component can be provided as a solid powder proton source component. Additionally or alternatively the solid proton source component can be provided as part of the dry wound dressing substrate (e.g. as proton source fibres).

The acid may be selected from one or more organic carboxylic acids or organic non-carboxylic reducing acids.

The expression “organic carboxylic acid” herein refers to any organic acid which contains one or more-COOH group in the molecule. An organic carboxylic acid may be straight-chain or branched. The carboxylic acid may be saturated or unsaturated. The carboxylic acid may be aliphatic or aromatic. The carboxylic acid may be acyclic or cyclic. The carboxylic acid may be a vinylogous carboxylic acid.

The organic carboxylic acid may carry one or more substituents, for example one or more hydroxyl group. Examples of hydroxyl-substituted organic carboxylic acids which may be used in the present disclosure include α-hydroxy-carboxylic acids, β-hydroxy-carboxylic acids and γ-hydroxy-carboxylic acids.

The expression “organic non-carboxylic reducing acid” herein refers to any organic reducing acid which does not contain a —COOH group in the molecule. An organic non-carboxylic reducing acid may be straight-chain or branched. The non-carboxylic reducing acid may be saturated or unsaturated. The non-carboxylic reducing acid may be aliphatic or aromatic. The non-carboxylic reducing acid may be acyclic or cyclic. The non-carboxylic reducing acid may be vinylogous.

The organic non-carboxylic reducing acid may carry one or more substituents, for example one or more hydroxyl group. Examples of hydroxyl-substituted organic non-carboxylic reducing acids which may be used in the present disclosure include the acidic reductones, for example reductic acid (2.3-dihydroxy-2-cyclopentanone).

The one or more organic carboxylic acid or non-carboxylic reducing acid may have a pKa₁ less than about 7.

The one or more organic carboxylic acid may comprise, consist of, or be one or more reducing carboxylic acids. The organic carboxylic acid may, for example, be selected from salicylic acid, acetyl salicylic acid, acetic acid, citric acid, glycolic acid, mandelic acid, tartaric acid, lactic acid, maleic acid, malic acid, benzoic acid, formic acid, propionic acid, α-hydroxypropanoic acid, β-hydroxypropanoic acid, β-hydroxybutyric acid, β-hydroxy-β-butyric acid, naphthoic acid, oleic acid, palmitic acid, pamoic (emboic) acid, stearic acid, malonic acid, succinic acid, fumaric acid, glucoheptonic acid, glucuronic acid, lactobioic acid, cinnamic acid, pyruvic acid, orotic acid, glyceric acid, glycyrrhizic acid, sorbic acid, hyaluronic acid, alginic acid, oxalic acid, salts thereof, and combinations thereof.

The organic carboxylic acid may be citric acid or a salt thereof.

The carboxylic acid may be or comprise a polymeric or polymerised carboxylic acid such as, for example, polyacrylic acid, polymethacrylic acid, a copolymer of acrylic acid and methacrylic acid, polylactic acid, polyglycolic acid, or a copolymer of lactic acid and glycolic acid. The term “organic carboxylic acid” used herein also cover partial or full esters of organic carboxylic acids or partial or full salts thereof, provided that those can serve as a proton source in use according to the present invention.

The organic non-carboxylic reducing acid may, for example, be selected from ascorbic acid; ascorbate palmitic acid (ascorbyl palmitate); ascorbate derivatives such as 3-O-ethyl ascorbic acid, other 3-alkyl ascorbic acids, 6-O-octanoyl ascorbic acid, 6-O-dodecanoyl ascorbic acid, 6-O-tetradecanoyl ascorbic acid, 6-O-octadecanoyl ascorbic acid and 6-O-dodecanedioyl ascorbic acid; acidic reductones such as reductic acid; erythorbic acid; salts thereof; and combinations thereof.

The organic non-carboxylic reducing acid may be ascorbic acid or a salt thereof.

The one or more organic carboxylic acid or organic non-carboxylic reducing acid of the proton source may suitably be present with the conjugate base thereof. The acid and its conjugate base may suitably form a buffer when contacted with or exposed to an aqueous environment. The acid and its conjugate base may be provided in a ratio to achieve the desired pH upon exposure to an aqueous environment.

The buffer system may be selected so that a desired pH is achieved upon exposure to an aqueous environment and maintained as the NOx generating reaction proceeds. The buffer system may be selected so that pH of the reaction may be in the range of about 3 to 9, for example about 4 to 8. For physiological contact or for contact with living cells and organisms, the pH of the reaction may be in the range of about 5 to about 8. The conjugate base, where present, may be added separately, or may be generated in situ from the proton source by adjustment of the pH using an acid and/or base, for example a mineral acid and/or a mineral base.

The proton source may be a citric acid/citrate buffer system, for example and citric acid/trisodium citrate buffer system.

The proton source may be or may comprise an acid precursor. An “acid precursor” is a species which can undergo a chemical reaction to provide an acid species. For example, the acid precursor may be a species which can undergo hydrolysis to provide an acid species. In other words, the acid precursor may be a hydrolysable acid precursor for releasing an acid on hydrolysis. For example, the acid precursor may be an ester. The acid precursor may be a photoacid. In other words, the acid precursor may be a species which become more acidic on absorption of light. For the avoidance of doubt, the term “photoacid” as used herein includes species that undergo reversible proton photodissociation and species that undergo irreversible proton photodissociation.

The solid proton source component can be provided as part of the dry wound dressing substrate (e.g. as proton source fibres). In some embodiments, the solid proton source component includes proton source fibres. In other words, the solid proton source component includes fibres capable of providing protons. Such proton source fibres include, but are not limited to, polyacrylic acid fibres (in particular partially neutralised polyacrylic acid fibres) and polyester fibres (in particular PLGA fibres).

In certain embodiments, the solid proton source component may include a combination of a solid powder proton source component with proton source fibres.

In particular embodiments, the solid proton source component includes a solid powder proton source component.

It is understood by the skilled person that the choice of acid component/proton source may be selected depending on the desired use.

### Combination of the Solid Powder Nitrite Salt Component and the Solid Proton Source Component

The solid powder nitrite salt component and the solid proton source component are present in the nitric oxide generating layer. Typically, the solid powder nitrite salt component and the solid proton source component will be present as a mixture of the components. In this way, the solid powder nitrite salt component and the solid proton source component are present in close proximity to react when exposed to an aqueous environment.

Where the nitric oxide generating layer includes the solid proton source component as part of the dry wound dressing substrate, the solid powder nitrite salt component and, when present, the solid powder proton source component may be combined by the methods described herein for combining solid powder components with the dry wound dressing substrate.

The mixture of the solid powder nitrite salt component and the solid powder proton source component may be achieved in a number of ways. The solid powder nitrite salt component and the solid powder proton source component may be added independently to the nitric oxide generating layer. In other words, the solid powder nitrite salt component may be added to the nitric oxide generating layer separately from the solid powder proton source component.

In particular embodiments, the solid powder nitrite salt component and the solid powder proton source component are mixed either before adding the components to other components of the nitric oxide generating layer or during the formation of the nitric oxide generating layer. There are various ways to mix the solid powder nitrite salt component and the solid powder proton source component. Particular ways of mixing the solid powder nitrite salt component and the solid powder proton source component are provided below.

### Combinations of Solid Powder Nitrite Salt Component and the Solid Powder Proton Source Component

The solid powder nitrite salt component and the solid powder proton source component may be provided by one or more particles that each contain a nitrite salt and a proton source. It is to be understood that the particles may contain the nitrite salt and the proton source within the same particle when the particles contain both the proton source and the nitrite salt.

Additionally or alternatively, the solid powder nitrite salt component and the solid powder proton source component may be provided by one or more particles that contain a nitrite salt and not a proton source and one or more particles that contain a proton source and not a nitrite salt. It is to be understood that the particles may contain either the nitrite salt or the proton source, and not the nitrite salt and proton source in the same particle. The one or more particles which contain either the nitrite salt or the proton source may be blended to provide a substantially homogeneous mixture of particles.

A “homogenous mixture” is a mixture that is uniform in composition such that it has the same proportion of its components throughout.

Therefore, the one or more particles containing either the nitrite salt or the proton source may be blended such as to provide a composition which has a uniform distribution and proportion of the nitrite salt particles and proton source particles.

The one or more particles may be present in the wound dressing as individual particles or as agglomerates of individual particles, or combinations thereof.

The expressions “agglomerate(s)”, “agglomeration” and “agglomerated together” herein refer to an aggregation or assemblage of primary (individual) particles exhibiting an identifiable collective behaviour.

In the present invention the agglomerates of individual particles may comprise (i) individual particles containing a nitrite salt and individual particles containing a proton source, (ii) individual particles containing a nitrite salt and a proton source, or (iii) combinations thereof and, optionally, a binding agent.

In the present invention, an identifiable collective behaviour may be resistance to mechanical separation, i.e., the particles adhesion to one another.

The particles or agglomerates of the solid powder nitrite salt component and the solid powder proton source component may be a suitable particle size for their desired use or application. For example, the particles or agglomerates of the solid composition may have a particle size of about 10 μm or less, for example, about 5 μm or less, about 4 μm or less, about 3 μm or less, about 2 μm or less or about 1 μm or less.

Alternatively, the particles or agglomerates solid powder nitrite salt component and the solid powder proton source component may have a particle size of greater than 5 μm. For example, the particles or agglomerates of the solid composition may have a particle size of greater than 50 μm, greater than 100 μm, greater than 250 μm, greater than 500 μm, greater than 750 μm, greater than 1000 μm.

The weight ratio of nitrite to proton source in the mixture of the solid powder nitrite salt component and the solid powder proton source component may be in the range of about 1:1 to about 1:99, such as in the range of about 1:4 to about 1:49 or about 1:7 to about 1:24.

The mixture of the solid powder nitrite salt component and the solid powder proton source component may comprise further optional additives, such as a binding agent (as above mentioned) or an organic polyol.

### Binding Agent

The mixture of the solid powder nitrite salt component and the solid powder proton source component may be substantially free of one or more binding agents. Alternatively, the mixture of the solid powder nitrite salt component and the solid powder proton source component may further include one or more binding agents.

A “binding agent” used herein refers to an agent that promotes the adhesion of particles, i.e. promotes the formation of an agglomeration of particles.

Suitable binding agents may include sugars, natural binders or synthetic or semisynthetic polymer binders. Sugar species may include, for example, sucrose or liquid glucose. Natural binders may include, for example, acacia, tragacanth, gelatin, starch paste, pregelatinized starch, alginic acid or cellulose. Synthetic or semisynthetic polymer binders may include, for example, methyl cellulose, ethyl cellulose, hydroxy propyl methyl cellulose (HPMC), hydroxy propyl cellulose, sodium carboxy methyl cellulose, polyvinylpyrrolidones (PVP), polyethylene glycols (PEG), polyvinyl alcohols, polymethacrylates. The binding agent may be a copolymer of 1-vinyl-2-pyrrolidone and vinyl acetate (copovidone). The binding agent may be microcrystalline cellulose.

The binding agent may be incorporated into the mixture of the solid powder nitrite salt component and the solid powder proton source component in % w/w of about 5% w/w to about 30% w/w. For example, the binding agent may be incorporated into the mixture of the solid powder nitrite salt component and the solid powder proton source component in a % w/w of about 10% w/w to about 25% w/w.

### Organic Polyol

The mixture of the solid powder nitrite salt component and the solid powder proton source component may be substantially free of one or more organic polyols. Alternatively, the mixture of the solid powder nitrite salt component and the solid powder proton source component may further include one or more organic polyol. When the mixture of the solid powder nitrite salt component and the solid powder proton source component includes one or more organic polyols, it is preferred that the organic polyol is added to the mixture of the solid powder nitrite salt component and the solid powder proton source component after any processing which involves removal of solvent (e.g., after spray drying or lyophilisation steps). In other words, the polyol may be added to a composition including one or more particles containing a nitrite salt and a proton source; or added to a mixture including one or more particles containing a nitrite salt and/or one or more particles containing a proton source (either before or after an agglomeration of these particles is formed).

The expression “organic polyol” herein refers to an organic molecule with two or more hydroxyl groups that is not a proton source, particularly for a nitrite salt reaction, and is not a saccharide or polysaccharide (the terms “saccharide” and “polysaccharide” include oligosaccharide, glycan and glycosaminoglycan). The organic polyol will thus have a pKa₁ of about 7 or greater.

The expression “organic polyol” herein preferably excludes reductants. Examples of reductants which are organic molecules with two or more hydroxyl groups and not a saccharide or polysaccharide are thioglycerol (for example, 1-thioglycerol), hydroquinone, butylated hydroquinone, ascorbic acid, ascorbate, erythorbic acid and erythorbate. Thioglycerol (for example, 1-thioglycerol), hydroquinone, butylated hydroquinone, ascorbate and erythorbate are thus preferably excluded from the expression “organic polyol” because they are reductants. Ascorbic acid and erythorbic acid are excluded from the expression anyway because they are proton sources, particularly for the nitrite salt reaction.

The organic polyol may be cyclic or acyclic or may be a mixture of one or more cyclic organic polyol and one or more acyclic organic polyol. For example, the one or more organic polyol may be selected from one or more alkane substituted by two or more OH groups, one or more cycloalkane substituted by two or more OH groups, one or more cycloalkylalkane substituted by two or more OH groups, and any combination thereof. The organic polyol may not carry any substituents other than OH.

The one or more organic polyol may be one or more acyclic organic polyol. The one or more acyclic organic polyol may be selected from the sugar alcohols having 4, 5, 6, 7, 8, 9, 10, 11 or 12 carbon atoms. The one or more acyclic organic polyol may be selected from the alditols, for example the alditols having 4, 5, 6, 7, 8, 9, 10, 11 or 12 carbon atoms. The one or more organic polyol may not include a saponin, sapogenin, steroid or steroidal glycoside.

Alternatively, the one or more organic polyol may be one or more cyclic organic polyol. The one or more cyclic organic polyol may be a cyclic sugar alcohol or a cyclic alditol. For example, the one or more cyclic polyol may be a cyclic sugar alcohol having 4, 5, 6, 7, 8, 9, 10, 11 or 12 carbon atoms or a cyclic alditol having 4, 5, 6, 7, 8, 9, 10, 11 or 12 carbon atoms. A specific example of a cyclic polyol is inositol.

The one or more organic polyol may have 7 or more hydroxy groups. The one or more organic polyol may be a sugar alcohol or alditol having 7 or more hydroxy groups. The one or more organic polyol may have 9 or more hydroxy groups. The one or more organic polyol may be a sugar alcohol or alditol having 9 or more hydroxy groups. The one or more organic polyol may have 20 or fewer hydroxyl groups. The one or more organic polyol may be a sugar alcohol or alditol having 20 or fewer hydroxy groups. The one or more organic polyol may have 15 or fewer hydroxyl groups. The one or more organic polyol may be a sugar alcohol or alditol having 15 or fewer hydroxyl groups. The one or more organic polyol may have a number of hydroxyl groups in the range of 7 to 20, for example, in the range of 9 to 15. The one or more organic polyol may include 9, 12, 15 or 18 hydroxy groups.

The one or more organic polyol may be a sugar alcohol compound comprising, for example consisting of, one or more monosaccharide units and one or more acyclic sugar alcohol units. The one or more organic polyol may be a sugar alcohol compound comprising, for example consisting of, a straight chain of one or more monosaccharide units and one or more acyclic sugar alcohol units or a branched chain of one or more monosaccharide units and one or more acyclic sugar alcohol units.

A “monosaccharide unit” as used herein refers to a monosaccharide covalently linked to at least one other unit (whether another monosaccharide unit or an acyclic sugar alcohol unit) in the compound. An “acyclic sugar alcohol unit” as used herein refers to an acyclic sugar alcohol linked covalently to least one other unit (whether a monosaccharide unit or another acyclic sugar alcohol unit) in the compound. The units in the compound may be linked through ether linkages. One or more of the monosaccharide units may be covalently linked to other units of the compound through a glycosidic bond. Each of the monosaccharide units may be covalently linked to other units of the compound through a glycosidic bond. The sugar alcohol compound may be a glycoside with a monosaccharide or oligosaccharide glycone and an acyclic sugar alcohol aglycone.

Acyclic sugar alcohol units may be sugar alcohol units having 4, 5, 6, 7, 8, 9, 10, 11 or 12 carbon atoms. The acyclic sugar alcohol unit may be selected from the group consisting of units of erythritol, threitol, arabitol, xylitol, ribitol, mannitol, sorbitol, galactitol, fucitol, iditol and volemitol.

One or more of the monosaccharide units may be a C₅ or C₆ monosaccharide unit, i.e., a pentose or hexose unit. Each monosaccharide unit may be a C₅ or C₆ monosaccharide unit. One or more of the sugar alcohol units may be a C₅ or C₆ sugar alcohol unit. Each sugar alcohol unit may be a C₅ or C₆ sugar alcohol unit.

The sugar alcohol compound may comprise, for example may consist of, n monosaccharide units and m acyclic sugar alcohol units, where n is a whole number and at least one, m is a whole number and at least one and (n+m) is no more than 10. The sugar alcohol compound may comprise, for example may consist of, a chain of n monosaccharide units terminated with one acyclic sugar alcohol unit, where n is a whole number between one and nine. The chain of monosaccharide units may be covalently linked by glycosidic bonds. Each monosaccharide unit may be covalently linked to another monosaccharide unit or the acyclic sugar alcohol unit by a glycosidic bond. The sugar alcohol compound may comprise, for example may consist of, a chain of 1, 2 or 3 monosaccharide units terminated with one acyclic alcohol unit. 1, 2, 3 or each monosaccharide unit may be a C₅ or C₆ monosaccharide unit. The acyclic alcohol unit may be a C₅ or C₆ sugar alcohol unit. Examples of the sugar alcohol compound include but are not limited to: isomalt, maltitol and lactitol (n=1); maltotriitol (n=2); and maltotetraitol (n=3).

Such sugar alcohol compounds may be described as sugar alcohols derived from a disaccharide or an oligosaccharide. “Oligosaccharide”, as used herein, refers to a saccharide consisting of three to ten monosaccharide units. Sugar alcohols derived from disaccharides or oligosaccharides may be synthesised (e.g. by hydrogenation) from disaccharides, oligosaccharides or polysaccharides (e.g. from hydrolysis and hydrogenation), but are not limited to compounds synthesised from disaccharides, oligosaccharides or polysaccharides. For example, sugar alcohols derived from a disaccharide may be formed from the dehydration reaction of a monosaccharide and a sugar alcohol. The one or more organic polyol may be a sugar alcohol derived from a disaccharide, trisaccharide or tetrasaccharide. Examples of sugar alcohols derived from disaccharides include but are not limited to isomalt, maltitol and lactitol. An example of a sugar alcohol derived from a trisaccharide includes but is not limited to maltotriitol. An example of a sugar alcohol derived from a tetrasaccharide includes but is not limited to maltotetraitol.

Organic polyols may be selected from erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, and any combination thereof. Glycerol can be used, and when present is preferably in association with one or more other organic polyol, for example erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, or any combination thereof.

Many organic polyols contain one or more chiral centre and thus exist in stereoisomeric forms. All stereoisomeric forms and optical isomers and isomer mixtures of the organic polyols are intended to be included within the scope of this invention. In particular, the D and/or L forms of all chiral organic polyols and all mixtures thereof may be used.

### Agglomeration of Particles

The agglomeration of particles may be achieved by any suitable means, known to the person of skill in the art.

The agglomeration of particles may be achieved by mechanical means, for example, by mechanically forcing the particles together. Agglomeration by mechanical means may be achieved by micronizing particles of a nitrite salt and particles of a proton source. Alternatively, agglomeration by mechanical means may be achieved by having particles that are substantially static-free.

The agglomeration of particles may be achieved by chemical means, for example, chemically facilitated adhesion or a chemical coating. Agglomeration by chemical means may be achieved by adhesion promoters, for example, moisture. Alternatively, agglomeration by chemical means may be achieved by a coating material that binds primary particles of a nitrite salt and primary particles of a proton source together. Suitable binding agents are previously discussed, and suitable coating materials are discussed in the section “Coated particles” below.

### Coated Particles

The one or more particles of the mixture of the solid powder nitrite salt component and the solid powder proton source component may be coated with an excipient (also referred to herein as coated particles).

The coated particles may include a single particle containing a nitrite salt and a proton source and coated with the excipient.

Alternatively, the coated particles may be an agglomeration of particles coated with the excipient and the agglomeration of particles includes (a) particles containing a nitrite salt and a proton source and/or (b) a mixture of one or more nitrite salt particles containing a nitrite salt and one or more proton source particles containing a proton source.

In this way, the coated particles include nitrite salt and proton source within the same coating.

The excipient may be hydrophobic. The excipient may be any material capable of coating the particles or agglomerates such that the particles or agglomerates are coated with a hydrophobic layer. The hydrophobic material may be a polymeric material, for example an organic polymeric material such as a polyol. The hydrophobic material may be an amphiphilic species, for example, a surfactant-type species such as a non-ionic, anionic, cationic or amphoteric surfactant-type species. The hydrophobic material may be an inorganic mineral material, for example, and inorganic mineral material that forms a 3D framework. The hydrophobic material may be biocompatible.

The hydrophobic material may include one or more of poly(lactic-co-glycolic acid) (PLGA), phospholipids, such as dipalmitoylphosphatidylcholine (DPPC), magnesium stearate, and mesoporous silica. The hydrophobic material may comprise the polymeric material poly(lactic-co-glycolic acid) (PLGA) without an acid end group or may comprise the polymeric material poly(lactic-co-glycolic acid) (PLGA) with an acid end group. The excipient may comprise a polyol, magnesium stearate, colloidal silica.

A “surfactant” as used herein refers to a surface-active agent which can lower the surface tension of a species in a medium or the interfacial tension between mediums. Surfactant species generally have a hydrophilic head and a hydrophobic tail.

The hydrophobic material may adhere to the particles or agglomerates by chemical bonding or by electrostatic or intermolecular forces.

The coating of the coated particles or coated agglomeration of particles may affect the reaction dynamics, for example the reaction kinetics, of the acidification of the nitrite salt when the coated particles or coated agglomerates are exposed to an aqueous environment.

The excipient may be a species capable of trapping or sequestering nitric oxide or nitric oxide precursors. For example, the excipient may comprise comprises thiols, alcohols, amines or amides.

The coated particles or coated agglomeration of particles of the mixture of the solid powder nitrite salt component and the solid powder proton source component may be a suitable particle size for the desired use or application. The coated particles or coated agglomeration of particles of the mixture of the solid powder nitrite salt component and the solid powder proton source component may have a particle size of about 10 μm or less, for example, about 5 μm or less, about 4 μm or less, about 3 μm or less, about 2 μm or less or about 1 μm or less. Alternatively, the coated particles or coated agglomerates of the mixture of the solid powder nitrite salt component and the solid powder proton source component may have a particle size of greater than about 5 μm. For example, the particles or agglomerates of the mixture of the solid powder nitrite salt component and the solid powder proton source component may have a particle size of greater than about 50 μm, greater than about 100 μm, greater than about 250 μm, greater than about 500 μm, greater than about 750 μm, greater than about 1000 μm.

Forming the Particles from a Mixture Containing a Nitrite Salt Solution and a Proton Source Solution

The mixture of the solid powder nitrite salt component and the solid powder proton source component may be formed by spray-drying or lyophilising a mixture containing a nitrite salt solution and proton source solution.

The particles of the mixture of the solid powder nitrite salt component and the solid powder proton source component may be formed from a mixture containing a nitrite salt solution and a proton source solution. Particles formed in this way should be formed by removal of solvent in a short time (e.g., thirty seconds or less) after mixing the nitrite salt solution and the proton source solution and/or the mixture is placed under reaction retarding conditions (e.g. at a temperature less than the freezing point of the solvent) after mixing nitrite salt solution and the proton source solution and for solvent removal. In this way, the solvent is removed from the mixture while minimising the acidification of the nitrite. An effective amount of nitrite and proton source may therefore be present in the resulting powder composition.

When the solvent is removed in a short time after mixing the nitrite salt solution and the proton source solution, the solvent may be removed in thirty second or less after the nitrite solution and proton source solution is mixed. In some examples, the solvent is removed in ten seconds or less, five seconds or less, two seconds or less or one second or less after mixing the nitrite solution and the proton source solution. In some examples, the solvent is removed in 500 milliseconds or less, 100 milliseconds or less, 50 milliseconds or less or 10 milliseconds or less after mixing the nitrite solution and the proton source solution.

In one example, the particles may be formed by spray-drying a mixture containing a nitrite salt solution and a proton source solution. Spray-drying of the mixture may allow the removal of solvent in a time of thirty seconds or less after mixing of the nitrite salt solution and the proton source solution. Spray-drying of materials is known per se.

The mixture is typically a mixture of an aqueous solution of the nitrite salt and an aqueous solution of the proton source. When aqueous solutions are used, the time between mixing the two aqueous solutions is minimised to suppress acidification of the nitrite salt. The aqueous solution of the nitrite salt and the aqueous solution of the acid may be mixed in line for about 1 to about 10 milliseconds, for example about 3 to about 5 milliseconds, before spray-drying takes place. Spray-drying may occur immediately after mixing of the nitrite and acid solutions. It is understood that mixing and spray-drying a mixture containing a nitrite salt solution and a proton source solution, as described, limits the potential reaction time between the proton source and nitrite component.

The particles formed by spray-drying the mixture containing a nitrite salt solution and an acid solution may have a particle size of about 10 μm or less, for example, about 5 μm or less, about 4 μm or less, about 3 μm or less, about 2 μm or less, or about 1 μm or less.

Spray-drying a mixture containing a nitrite salt solution and an acid solution as described may result in a mixture of the solid powder nitrite salt component and the solid powder proton source component where each particle contains nitrite salt and proton source components.

Particles formed by spray-drying a mixture containing a nitrite salt solution and a proton source solution may be any suitable morphology. For example, particles formed by spray-drying a mixture containing a nitrite salt solution and proton source solution may be crystalline in form or amorphous in form. The particles formed by spray-drying a mixture containing a nitrite salt solution and a proton source solution may be amorphous in form.

Additionally or alternatively, the mixture of nitrite salt solution and proton source solution is placed under a reaction-retarding condition (e.g. at a temperature less than the freezing point of the solvent) before, during or immediately after mixing the nitrite salt solution and the proton source solution and for solvent removal. In this way, the acidification of the nitrite is retarded until the solvent is removed. In particular, the solvent may be an aqueous solvent.

A particular example of a reaction-retarding condition is a temperature of the mixture below the freezing point of the solvent. In this way, the reaction rate of the acidification of nitrite may be slowed while the solvent is removed. Where the temperature of the mixture is below the freezing point of the solvent, the nitrite solution and the proton source solution are typically mixed at a temperature above the freezing point of the solvent before the temperature of the mixture is reduced to below the freezing point of the solvent. In this way, good mixing of the solutions may occur.

In some examples, the solvent removal may occur at a reduced gas pressure. In particular, the solvent removal may occur at a reduced gas pressure in combination at a temperature below the freezing point of the solvent to be removed.

A particularly useful technique to remove the solvent under a reaction-retarding condition is lyophilisation (also referred to as “freeze-drying”).

It should be noted that the terms “removal of solvent” and/or “drying” as used herein to achieve a solid powder composition. These terms include but are not limited to the complete removal of solvent. In some examples, a solid powder composition may include trace amounts of residual solvent. For example, the powder composition may contain up to about 10% of residual solvent, for example up to about 5% residual solvent, up to about 3% residual solvent or up to about 1% residual solvent. Additional drying techniques, such as vacuum drying, may be employed after the initial removal of solvent in order to provide the solid powder composition.

### Combining Solids to Form an Agglomeration of Particles

The mixture of the solid powder nitrite salt component and the solid powder proton source component may be formed by combining a nitrite-containing solid with a proton source-containing solid to form an agglomeration of particles, wherein the agglomeration of particles includes one or more particles containing a nitrite salt and one or more particles containing a proton source.

Combining a nitrite-containing solid with a proton source-containing solid to form an agglomeration of particles may be achieved, for example, by (a) blending one or more nitrite salt particles and one or more proton source particles, wherein the nitrite salt particles are formed by spray-drying a nitrite salt solution and the proton source particles are formed by spray-drying proton source solution; or (b) forming one or more particles by micronizing a nitrite salt solid with a proton source solid.

### Blended Spray-Dried Nitrite Particles and Spray-Dried Acid Particles

The mixture of the solid powder nitrite salt component and the solid powder proton source component may be formed by:
(i) spray-drying or lyophilising a solution containing a nitrite salt,
(ii) spray-drying or lyophilising a solution containing a proton source,
(iii) blending the species of (i) and (i).

The mixture of the solid powder nitrite salt component and the solid powder proton source component may be a blend of nitrite salt particles and proton source particles, wherein the nitrite salt particles are formed by spray-drying a nitrite salt solution and the proton source particles are formed by spray-drying a proton source solution. The spray-dried nitrite salt particles and the spray-dried proton source particles may be blended by standard means known to a person of skill in the art to provide a blended solid powder composition.

The spray-dried nitrite particles and the spray-dried proton source particles may be blended at a nitrite to proton source weight ratio of about 1:1 to about 1:99, such as in the range of about 1:4 to about 1:49 or about 1:7 to about 1:24.

The spray-dried particles of nitrite salt and the spray-dried particles of proton source may be blended for a time of, about 5 to about 60 minutes, for example a time of about 10 to about 40 minutes, or a time of about 15 to about 30 minutes. The spray-dried particles of nitrite salt and the spray-dried particles of proton source may be blended for a time of about 20 minutes.

The particles formed by a nitrite salt solution and spray-drying an acid solution and blending these components as described may have a particle size of about 10 μm or less, for example, about 5 μm or less, about 4 μm or less, about 3 μm or less, about 2 μm or less, or about 1 μm or less.

Spray-drying a nitrite salt solution and spray-drying a proton source solution and blending these components as described may result in a mixture of the solid powder nitrite salt component and the solid powder proton source component including an agglomeration of particles, wherein the agglomeration includes one or more particles containing nitrite salt and one or more particles containing proton source.

Particles formed by spray-drying a nitrite salt solution and spray-drying a proton source solution and blending these components may be any suitable morphology. For example, particles formed by spray-drying a nitrite salt solution and spray-drying proton source solution and blending these components may be crystalline in form or amorphous in form. The particles formed by spray-drying a mixture containing a nitrite salt solution and proton source solution may be amorphous in form.

Particles Formed from Micronizing a Nitrite Salt Solid with an Acid Solid

The particles may be formed by micronizing a nitrite salt solid with a proton source solid.

The expression “micronizing” as used herein refers to a process for reducing the average particle size of a solid composition, typically to within the micrometre scale. Micronizing can be achieved by standard processes known to a person of skill in the art. For example, micronizing may occur by milling or grinding the particles or by utilisation of super critical fluids.

Where the proton source is a buffered acid system, the proton source solid may be two components, a solid acid component and a solid conjugate base component. The nitrite salt solid and the proton source solid may be micronized in a ratio of about 1:1 to about 1:99, such as in the range of about 1:4 to about 1:49 or about 1:7 to about 1:24, e.g. 1:9 w/w nitrite:proton source.

The particles formed by micronizing a nitrite salt solid with a proton source solid may have a particle size of about 10 μm or less, for example, about 5 μm or less, about 4 μm or less, about 3 μm or less, about 2 μm or less, or about 1 μm or less.

Micronizing a nitrite salt solution with a proton source solution as described may result in a solid powder composition of particles containing nitrite salt and particles containing proton source. Micronizing a nitrite salt solution with a proton source solution as described may result in a solid powder composition which comprises agglomerates comprising particles containing nitrite salt and particles containing proton source.

Particles formed by micronizing a nitrite salt solution with a proton source solution may be any suitable morphology. For example, particles formed by micronizing a nitrite salt solution with a proton source solution may be crystalline in form or amorphous in form. The particles formed by micronizing a nitrite salt solution with a proton source solution may be crystalline in form.

The particles formed by micronizing may include one or more of the optional additives (in addition to the proton source and nitrite salt) as described above. In particular, the particles formed by micronizing may include a binding agent as described above. The binding agent may be micronized with the nitrite solid and the proton source solid.

### Other Features of the Wound Dressing

The wound dressing may be a one-part wound dressing. One-part wound dressings include all of the components required to apply the wound dressing to a subject in a single piece. In this way, the wound dressing does not require assembly by a practitioner before applying to a subject.

The wound dressing may be a single layer (the nitric oxide generating layer) wound dressing or may be a multi-layer wound dressing (including the nitric oxide generating layer).

The wound dressing may, in particular, include a backing layer. The backing layer is typically positioned on an exterior face of the wound dressing and on the opposing side to a face of the wound dressing adapted to be applied to a subject. In this way, the backing layer may protect the wound and active components of the wound dressing when applied from the environment. The backing layer may be flexible. The backing layer may be permeable or semi-permeable to gases. The backing layer may be made from polyurethane. The backing layer may include an adhesive for attaching the wound dressing to a subject. Backing layers for wound dressings are known per se.

The wound dressing may include a removable protective layer on the exterior face or faces of the wound dressing for protecting the wound dressing components (e.g. the nitric oxide generating layer) before application of the wound dressing to a subject. The removable protective layer or layers are typically removed from the wound dressing before application of the wound dressing to a subject to expose the active wound dressing components (e.g. the nitric oxide generating layer) to the wound. The removable protective layer or layers may be flexible. The removable protective layer or layers may be transparent or semi-transparent.

The nitric oxide generating layer may be intended to be applied directly to a wound of a subject, when in use. The wound dressing may be configured such that the nitric oxide generating layer is applied directly to the wound of a subject, in use. For example, the nitric oxide generating layer may form an exterior face of the wound dressing. Alternatively, the nitric oxide generating layer is adjacent to an outer removable protective film or layer for removing prior to direct application of the nitric oxide generating layer to the wound of a subject. In other words, the wound dressing may include a removable protective film forming an exterior face of the wound dressing, and wherein the nitric oxide generating layer is adjacent to the removable protective film. In this way, the removable protective film can be removed prior to application and the nitric oxide generating layer can be applied directly to the wound of a subject.

Alternatively, one or more permeable layers may be adjacent to the nitric oxide generating layer and the one or more permeable layers be intended to be applied to the wound dressing. The wound dressing may include one or more permeable layers adjacent to the nitric oxide generating layer and be configured such that the one or more permeable layers are applied directly to a wound of subject, in use. The wound dressing may further include an outer removable protective film or layer for removing prior to direct application of the one or more permeable layers to the wound of a subject. In other words, the wound dressing may include a removable protective film forming an exterior face of the wound dressing, wherein the one or more permeable films are adjacent to the removable protective film, and the one or more permeable layers adjacent to the nitric oxide generating layer. In this way, the removable protective film can be removed prior to application and one or more permeable layers adjacent to the nitric oxide generating layer can be applied directly to the wound of a subject. The one or more permeable layers may be made from any permeable material, typically any gas and/or liquid permeable material. In this way, nitric oxide may enter these layers and/or liquid may pass through these layers into the nitric oxide generating layer.

The wound dressing may include one or more further dry layers adjacent to nitric oxide generating layer. The water content of any layer adjacent to the nitric oxide generating layer may be 10% or less, 5% or less, 2% or less or 1% or less based on the weight of the layer adjacent to the nitric oxide generating layer.

The material of the additional layer(s) of the wound dressing may be mesh (woven or non-woven), non-woven bat, film, foam, alginate, amorphous hydrogel, crosslinked hydrogel or a membrane. The layer(s) of the wound dressing may be formed from natural or synthetic materials, for example, the layer(s) of the wound dressing may be carboxymethylcellulose fibres and synthetic polymer fabrics. The present invention is not limited to the uses and materials listed above and other suitable materials and uses of wound dressing would be known to the skilled person.

### Further Anti-Microbials

The acidification of the nitrite salt component and the proton source component typically has anti-microbial activity. In some examples, the wound dressing includes a further anti-microbial. Anti-microbials are known per se. In some examples, the wound dressing includes AgNO₂ as both the anti-microbial and the nitrite salt.

### Packaged Wound Dressings

The present invention also provides a packaged wound dressing comprising a wound dressing as described herein within a low moisture permeability packaging.

The low moisture permeability packaging may include one or more low moisture permeability materials (e.g. aluminium foil) in the walls of the packaging. In particular embodiments, the low moisture permeability packaging includes one or more low moisture permeability materials (e.g. aluminium foil) in the walls of the packaging and the wound dressing and is be hermetically sealed. The low moisture permeability packaging may include one or more low moisture permeability materials (e.g. aluminium foil) in at least part of all of the exterior walls of the packaging.

The packaging atmosphere within the packaged wound dressing may have a low moisture content at initial packaging. The packaging atmosphere may have a relative humidity of 30% or less, 25% or less, 20% or less, 15% or less or 10% or less. Relative humidity can be measured using a hygrometer.

The packaging atmosphere may include an inert packaging gas, such as nitrogen, argon, helium or CO₂. The packaging atmosphere includes 10% or less, 8% or less, 5% or less, 2% or less, 1% or less oxygen. In some embodiments, the packaging atmosphere is substantially free of oxygen.

Additionally or alternatively, the package may include one or more pack inserts that sequester moisture. Such pack inserts may be desiccant packs, such as silica gel packs.

### Methods of and Combinations for Treating a Wound

The present invention provides a method of treating a wound, the method comprising applying a wound dressing as described herein to wound of a subject. The wound dressing may be a one-part wound dressing. In other words, the wound dressing may be provided as a single piece prior to the point of need.

The present invention also provides a combination of a solid powder nitrite salt component and a solid proton source component in a wound dressing as described herein for use in treating a wound in a subject. The wound dressing may be a one-part wound dressing. In other words, the wound dressing may be provided as a single piece prior to the point of need.

In some embodiments, the method includes applying the wound dressing as packaged to the wound of a subject. In other embodiments, the method includes removing a removable outer layer or film of the wound dressing before applying to the wound of a subject. Typically, the method will not include combining two or more parts of the wound dressing before application to the wound of a subject.

In some embodiments, the method includes adding water (including aqueous solutions, suspensions, gels or other forms including water) to the nitric oxide generating layer prior to applying the wound dressing to the wound of a subject. The addition of water may be directly to the nitric oxide generating layer or may be indirectly to the nitric oxide generating layer (e.g. through one or more permeable layers adjacent to the nitric oxide generating layer). The added water may be a sterile aqueous solution. The aqueous environment may be a sterile saline solution.

Alternatively, the wound dressing is applied to the wound dressing of a subject without addition of water. In this way, aqueous fluids from the subject (e.g. blood and/or exudate) may be absorbed by the nitric oxide generating layer of the wound dressing and activate the generation of nitric oxide.

The subject may be a human or animal subject. The subject may be a human or a domesticated animal.

### Methods of Producing Solid Powder Components

### Method of Producing a Solid Powder Composition by Solvent Removal

The method of making the mixture of the solid powder nitrite salt component and the solid powder proton source component may include removing solvent from a mixture of a nitrite solution and a proton source solution in such a way so as to minimise acidification before a solid powder composition forms.

In one example, the method includes the step of removing the solvent in less than thirty seconds (e.g. by spray-drying) after mixing of a nitrite solution and a proton source solution to form the solid.

In another example, the method includes providing reaction-retarding conditions (e.g. lyophilisation) and during solvent removal and before, during and/or immediately after mixing a nitrite salt solution and a proton source solution.

In one example, the method may include the step of removing the solvent from an aqueous mixture containing a nitrite salt solution and a proton source solution to form the solid powder.

The aqueous solution of the nitrite salt may have a concentration in the range of about 0.1 M to about 5 M. The aqueous solution of the nitrite salt may have a concentration of at least about 0.1 M, at least about 0.2 M, at least about 0.5 M, at least about 0.75 M, or at least about 1 M. The aqueous solution of the nitrite salt may have a concentration of up to about 5 M, up to about 4 M, up to about 3 M or up to about 2 M. For example, the aqueous solution of the nitrite salt may have a concentration in the range of about 1 M to about 2 M, such as about 1.5 M. The aqueous solution of the nitrite salt may have a pH of about 6.5 to about 9, for example, from about 7 to about 8.

The aqueous solution of the proton source may have a concentration in the range of about 0.1 M to about 5 M. The aqueous solution of the nitrite salt may have a concentration of at least about 0.1 M, at least about 0.2 M, at least about 0.5 M, at least about 0.75 M, or at least about 1 M. The aqueous solution of the nitrite salt may have a concentration of up to about 5 M, up to about 4 M, up to about 3 M or up to about 2 M.

For example, the aqueous solution of the nitrite salt may have a concentration in the range of about 0.5 M to about 1.5 M, such as about 1 M. The aqueous solution of the citric acid may have a pH of about 4 to 6. The pH of the aqueous solution of the proton source may be adjusted using, for example a mineral base such as sodium hydroxide.

In some examples, the step of removing the solvent takes 20 seconds or less, ten seconds or less, five seconds or less, two seconds or less or one second or less after mixing the nitrite solution and the proton source solution. In some examples, the solvent is removed in 500 milliseconds or less, 100 milliseconds or less, 50 milliseconds or less or 10 milliseconds or less after mixing the nitrite solution and the proton source solution.

### Spray Drying

The mixture of the solid powder nitrite salt component and the solid powder proton source component may be produced by spray-drying a nitrite solution and a proton source solution.

The aqueous solution of the nitrite salt and the aqueous solution of the acid may be mixed in line for about 1 to about 10 milliseconds, for example about 3 to about 5 milliseconds, before spray-drying takes place. Spray-drying may occur immediately after mixing of the nitrite and proton source solutions. It is understood that mixing and spray-drying a mixture containing a nitrite salt solution and a proton source solution, as described, greatly limits the potential reaction time between the proton source and nitrite component and halts the reaction entirely upon the rapid removal of moisture.

The spray-drying may occur at an outlet temperature in the range of about 60 to about 80° C., such as about 65 to about 75° C. or about 68 to about 70° C. The spray-drying may occur at an atomisation pressure in the range of about 1 to 6 bar. The spray-drying may occur at a liquid feed rate in a range of about 1 to about 5 g/min, such as about 2 g/min to about 4 g/m, or about 3 g/min.

### Reaction-Retarding Condition(s)

As an alternative, the method may include providing reaction-retarding conditions (e.g., lyophilisation) and during solvent removal and before, during and/or immediately after mixing a nitrite salt solution and a proton source solution.

A particular example of a reaction-retarding condition is a temperature of the mixture below the freezing point of the solvent. In this way, the reaction rate of the acidification of nitrite may be slowed while the solvent is removed. Where the temperature of the mixture is below the freezing point of the solvent, the nitrite solution and the proton source solution are typically mixed at a temperature above the freezing point of the solvent before the temperature of the mixture is reduced to below the freezing point of the solvent. In this way, good mixing of the solutions may occur.

In some examples, the solvent removal may occur at a reduced gas pressure. In particular, the solvent removal may occur at a reduced gas pressure in combination at a temperature below the freezing point of the solvent to be removed.

A particularly useful technique to remove the solvent under a reaction-retarding condition is lyophilisation (also referred to as “freeze-drying”).

The time taken to remove solvent after mixing the nitrite solution and the proton source solution under the retarded-reaction conditions may be about 10 minutes or less. Under these conditions, it may be less important to remove the solvent (e.g. water) so rapidly. However, removal of solvent in a relatively short time frame is also desired to further limit acidification of the nitrite. In some examples, the solvent is removed under reaction-retarding conditions in about 8 minutes or less, for example, about 7 minutes or less, about 6 minutes or less, about 5 minutes or less, about 4 minutes or less, about 3 minutes or less or about 2 minutes or less after mixing the nitrite solution and the proton source solution. In further examples, the step of removing the solvent takes about 1 minute or less, about 30 seconds or less, about 20 seconds or less, about 15 seconds or less or about 10 second or less after mixing the nitrite solution and the proton source solution.

It should be noted that the terms “removal of solvent” and/or “drying” as used herein to achieve a solid powder composition. These terms include but are not limited to the complete removal of solvent. In some examples, a solid powder composition may include trace amounts of residual solvent. For example, the powder composition may contain up to about 10% of residual solvent, for example up to about 5% residual solvent, up to about 3% residual solvent or up to about 1% residual solvent. Additional drying techniques, such as vacuum drying, may be employed after the initial removal of solvent in order to provide the solid powder composition.

### Method of Combining Particles to Form an Agglomeration of Particles

Forming an agglomeration of particles including particles containing a nitrite salt and particles containing a proton source can be achieved in a number of ways.

In one example, the method may include the steps of:
(i) Spray-drying or lyophilising a nitrite salt solution to form nitrite salt particles;
(ii) Spray-drying or lyophilising a proton source solution to form proton source particles; and
(iii) Blending the nitrite salt particles and the proton source particles.

The aqueous solution of the nitrite salt may have a concentration in the range of about 0.1 M to about 5 M. The aqueous solution of the nitrite salt may have a concentration of at least about 0.1 M, at least about 0.2 M, at least about 0.5 M, at least about 0.75 M, or at least about 1 M. The aqueous solution of the nitrite salt may have a concentration of up to about 5 M, up to about 4 M, up to about 3 M or up to about 2 M. For example, the aqueous solution of the nitrite salt may have a concentration in the range of about 1 M to about 2 M, such as about 1.5 M. The aqueous solution of the nitrite salt may have a pH of about 6.5 to about 9, for example, from about 7 to about 8.

The aqueous solution of the proton source may have a concentration in the range of about 0.1 M to about 5 M. The aqueous solution of the nitrite salt may have a concentration of at least about 0.1 M, at least about 0.2 M, at least about 0.5 M, at least about 0.75 M, or at least about 1 M. The aqueous solution of the nitrite salt may have a concentration of up to about 5 M, up to about 4 M, up to about 3 M or up to about 2 M. For example, the aqueous solution of the nitrite salt may have a concentration in the range of about 0.5 M to about 1.5 M, such as about 1 M. The aqueous solution of the citric acid may have a pH of about 4 to 6. The pH of the aqueous solution of the proton source may be adjusted using, for example a mineral base such as sodium hydroxide.

The spray-drying may occur at an outlet temperature in the range of about 60 to about 80° C., such as about 65 to about 75° C. or about 68 to about 70° C. The spray-drying may occur at an atomisation pressure in the range of about 1 to 6 bar. The spray-drying may occur at a liquid feed rate in a range of about 1 to about 5 g/min, such as about 2 g/min to about 4 g/m, or about 3 g/min.

In some examples, the spray-dried particles are further dried, for example, by vacuum drying.

The spray-dried or lyophilised nitrite salt particles and the spray-dried or lyophilised proton source particles may be blended by standard means known to a person of skill in the art to provide a blended solid powder composition.

The spray-dried or lyophilised nitrite particles and the spray-dried or lyophilised proton source particles may be blended at a weight ratio of nitrite to proton source in the range of about 1:1 to about 1:99, such as in the range of about 1:4 to about 1:49 or about 1:7 to about 1:24.

The spray-dried particles of nitrite salt and the spray-dried particles of proton source may be blended for a time of, about 5 to about 60 minutes, for example a time of about 10 to about 40 minutes, or a time of about 15 to about 30 minutes. The spray-dried particles of nitrite salt and the spray-dried particles of proton source may be blended for a time of about 20 minutes.

### Method of Producing the Mixture of the Solid Powder Nitrite Salt Component and the Solid Powder Proton Source Component by Micronization

A method of producing mixture of the solid powder nitrite salt component and the solid powder proton source component may include the step of micronizing a nitrite salt solid with a proton source solid to produce a solid powder composition. Micronization is known per se. Micronizing can be achieved by standard processes known to a person of skill in the art. For example, micronizing may occur by milling or grinding the particles or utilisation of super critical fluids.

The nitrite salt solid may be micronized with the proton source solid for a time of about 5 to about 30 minutes, for example about 5 to about 20 minutes, or from about 5 to about 15 minutes. The nitrite salt solid may be micronized with the proton source solid for a time of about 10 minutes.

The nitrite salt solid may be micronized with the proton source solid with a venturi pressure of 8 bar and a grinding pressure of 2 bar.

The present inventors have found that micronizing the nitrite solid with (i.e. at the same time as) the proton source solid may produce solid powder compositions with better release of nitric oxide when exposed to an aqueous environment than solid powder compositions formed by blending of separately micronized nitrite powders and separately micronized proton source powders.

Methods of Producing Solid Powder Compositions with Coated Particles

A mixture of the solid powder nitrite salt component and the solid powder proton source component may be produced comprising particles coated in a hydrophobic material.

The method may include the step of either:
(i) Coating particles containing a nitrite salt and a proton source with a hydrophobic material; or
(ii) Combining one or more nitrite salt particles containing a nitrite salt and one or more proton source particles containing a proton source and then coating the mixture.

The hydrophobic material may be the same hydrophobic material as described above.

The particles or agglomeration of particles may be coated in any suitable manner known to the person of skill in the art.

The particles or agglomeration of particles may be coated by dispersing the particles or agglomerates in a solution containing a hydrophobic material and drying the solution to provide particles or agglomeration of particles that are coated with a layer of the hydrophobic material. In some examples, the solution includes a non-polar solvent. In particular examples, the solution is free of polar solvent (e.g. methanol). Such polar solvents may dissolve at least part of the particle. In particular, the solution may be aqueous-free.

The hydrophobic material may, for example, be PLGA. The particles or agglomeration of particles may be dried at a 1:1 w/w ratio with the hydrophobic material. The solution which the particles or agglomeration of particles are dispersed or suspended in may be a solution of DCM and the hydrophobic material.

In particular embodiments, the suspension of particles in the hydrophobic material solution is dried by spray drying. The solution containing the hydrophobic material in which the particles or agglomeration of particles are dispersed in may be spray-dried at an outlet temperature of about 28 to 30° C. The solution containing the hydrophobic material which the particles or agglomerates are dispersed in may be spray-dried at an atomisation pressure of about 1 bar. The solution containing the hydrophobic material which the particles or agglomerates are dispersed in may be spray-dried a liquid feed rate of about 2 g/min.

The coated particles or coated agglomeration of particles may have a particle size of less than about 10 μm, for example less than about 9 μm, for example less than about 8 μm, less than about 7 μm, less than about 6 μm, or less than about 5 μm.

The particles or agglomeration of particles may be coated by blending the particles or agglomeration of particles with the hydrophobic material to provide particles or agglomerates that are coated with a layer of the hydrophobic material. The hydrophobic material may, for example, be DPPC, magnesium stearate, mesoporous silica or combinations thereof. The particles or agglomerates may be blended at a ratio of 1:1 w/w with the hydrophobic material. The hydrophobic material may be sieved prior to blending. Alternatively, the hydrophobic material may not be sieved prior to blending.

The particles or agglomeration of particles may be blended with the hydrophobic material for a time of about 10 to about 40 minutes, or a time of about 15 to about 30 minutes. The spray-dried particles of nitrite salt and the spray-dried particles of proton source may be blended for a time of about 20 minutes.

### Aqueous Environment

The nitric oxide generating layers of the present invention typically release NOx when in contact with an aqueous environment. The aqueous environment is not particularly limited.

The aqueous environment may be an aqueous biological fluid, such as a bodily fluid. Such bodily fluids may include wound discharge or exudate and/or blood (such as blood plasma, blood serum).

Alternatively, the aqueous environment may be a sterile aqueous solution. The aqueous environment may be a saline solution.

In some embodiments the solid powder compositions may be sufficiently hygroscopic to absorb moisture from air, which is sufficient to start the release of NOx.

### EXAMPLES

### Preparation of Solid Powder Compositions

### Materials and Analytical Methods

The following materials were obtained from commercial sources: sodium nitrite from Honeywell, citric acid from Sigma Aldrich, trisodium citrate from Merck, sodium hydroxide from Fisher, PLGA RG 502 H from Sigma Aldrich, mesoporous silica (Syloid 244FP) from Grace, dipalmitoyl phosphatidylcholine (DPPC) from Avanti, Kollidon VA64 Fine from BASF, microcrystalline cellulose from JRS Pharma and dichloromethane (DCM) from Sigma Aldrich. Deionised (DI) water (18.2 MΩ) was prepared using an ELGA water purification system.

Unless stated otherwise, the following analytical methods were used.

### Dry Powder Particle Size Distribution (PSD) by Sympatec

Laser particle size analysis of spray dried powders was performed using a Sympatec HELOS particle size analyser equipped with an R3 lens (0.5-175.0 μm range)/R5 lens (0.5-875.0 μm range) and an ASPIROS dispersion unit. Dispersal was achieved using compressed air at a pressure of 3.00 bar and a depression of 60 mbar. ASPIROS glass tubes were filled with powder in a reduced humidity environment (<25% RH) and sealed with Parafilm until the measurement was taken. Measurements were made in triplicate unless stated and the mean data was reported.

### Example 1: Spray-Drying a Mixture Containing a Nitrite Salt Solution and a Proton Source Solution to Form the Solid Powder Composition

A feed solution of 1.5M sodium nitrite (feed solution 1) was prepared by dissolving the required sodium nitrite mass in deionised water. A feed solution of 1M citric acid (feed solution 2), adjusted to pH 4, was prepared by dissolving the required citric acid mass in deionised water and adjusting the pH to 4 using 10M aqueous sodium hydroxide solution. The pH of the solution was measured using a Mettler Toledo Seven Compact PH meter.

Feed solutions 1 and 2 were spray dried using a Buchi B290 spray dryer, fitted with a Buchi two-fluid nozzle. The two feed solutions were pumped simultaneously using separate feed lines (platinum-cured silicone L/S 14 tubing) connected using a Y-piece fitting and a single Masterflex peristaltic pump, which combined the feed solutions immediately prior to atomisation. A standard Buchi cyclone and collection pot were fitted for product collection.

The feed solutions were spray dried in two batches, with the following conditions:

| | Outlet | Atomisation | Liquid Feed |
|---|---|---|---|
| | Temperature | pressure | rate |
| Example | (° C.) | (bar) | (g/min) |
| 1A | 68-70 | 5.5 | 3.06 |
| 1B | 68-70 | 1.5 | 3.05 |

Both batches were then vacuum dried using an Edwards Super Modulyo freeze dryer set to 25° C. for 24 hours.

Particle size distribution measurements were then taken for both batches using a Sympatec HELOS particle size analyser equipped with an R3 lens (0.5-175.0 μm range) and a ASPIROS dispersion unit. Dispersal was achieved using compressed air at a pressure of 3.00 bar and a depression of 60 bar. Measurements were made in triplicated.

The resultant particles size distribution measurements were as follows:

| | Mean Particle size (n = 3) by laser diffraction (μm) | | | |
|---|---|---|---|---|
| Example | X₁₀ | X₅₀ | X₉₀ | VMD |
| 1A | 1.27 | 2.92 | 5.96 | 3.32 |
| 1B | 1.83 | 3.84 | 7.54 | 4.35 |

VMD = volume mean diameter

### Example 2: Spray-Drying a Nitrite Salt and Proton Source Separately, and then Blending to Produce the Solid Composition

A solution of 1.5 M sodium nitrite was prepared by dissolving the required sodium nitrite mass in deionised water.

A solution of 1 M citric acid, adjusted to pH 5.6, was prepared by dissolving the required citric acid mass in deionised water and adjusting the pH to 5.6 using 10 M aqueous sodium hydroxide solution. The pH of the solution was measured using a Mettler Toledo Seven Compact PH meter.

These feed solutions were spray dried separately using the Buchi B290 spray dryer, under the following conditions:

| | | Outlet | Atomisation | Liquid |
|---|---|---|---|---|
| | | Temperature | pressure | feed rate |
| Component | Feed solution | (° C.) | (bar) | (g/min) |
| 2A | 1.5M sodium | 69 | 5.5 | 2.94 |
| | nitrite | | | |
| 2B | 1M citric acid/ | 69-70 | 5.5 | 3.00 |
| | sodium citrate | | | |
| | solution (pH 4) | | | |
| 2C | 1M citric acid/ | 69-70 | 5.5 | 3.02 |
| | sodium citrate | | | |
| | solution (pH 5.6) | | | |

All batches were then vacuum dried using an Edwards Super Modulyo freeze dryer set to 25° C. for 24 hours.

Particle size distribution measurements were then taken for the three batches using a Sympatec HELOS particle size analyser equipped with an R3 lens (0.5-175.0 μm range) and a ASPIROS dispersion unit. Dispersal was achieved using compressed air at a pressure of 3.00 bar and a depression of 60 bar. Measurements were made in triplicated.

| | Mean Particle size (n = 3) by laser diffraction (μm) | | | |
|---|---|---|---|---|
| Component | X₁₀ | X₅₀ | X₉₀ | VMD |
| 2A | 1.63 | 3.31 | 6.10 | 3.68 |
| 2B | 0.85 | 2.85 | 7.11 | 3.64 |
| 2C | 0.95 | 3.87 | 9.16 | 4.65 |

The spray-dried nitrite solid (component 2A) and the spray-dried citric acid solid at pH 5.6 (component 2C) were then blended in a ratio of 9:1 w/w citrate solid:nitrite solid, using a Turbula T2F mixer at 46 rpm for 20 minutes, resulting in the powder composition of Example 2.

### Example 3: Micronizing a Nitrite Salt Solid with Proton Source Solid to Produce the Solid Powder Composition

Sodium nitrite, citric acid and trisodium citrate were combined together in the following weight proportions: 10.79%, 14.74% and 74.47%, respectively. The mixture was blended at 47 rpm for 10 min using a Turbula T2F mixer.

The blend was micronised using an Atritor M3 fluid energy mill with a venturi pressure of 8 bar and grinding pressure of 2 bar. The blend was fed directly into the hopper at a target feed rate of ˜2 g/min. The produced powder (Example 3) was collected into a single collection jar under reduced humidity (20% RH).

Particle size distribution measurements were then taken using a Sympatec HELOS particle size analyser equipped with an R3 lens (0.5-175.0 μm range) and a ASPIROS dispersion unit. Dispersal was achieved using compressed air at a pressure of 3.00 bar and a depression of 60 bar. Measurements were made in triplicated.

The resultant particles size distribution measurements were as follows:

| | Mean Particle size (n = 3) by laser diffraction (μm) | | | |
|---|---|---|---|---|
| Example | X₁₀ | X₅₀ | X₉₀ | VMD |
| 3 | 0.68 | 1.60 | 3.63 | 1.93 |

VMD = volume mean diameter

### Reference Example 4: Micronizing a Nitrite Salt and Proton Source Separately, and then Blending to Product the Solid Composition

Sodium nitrite was micronised using an Atritor M3 fluid energy mill with a venturi pressure of 8 bar and grinding pressure of 2 bar. The sodium nitrite was fed directly into the hopper at a target feed rate of ˜2 g/min. The produced powder (component 4A) was collected into a single collection jar under reduced humidity (20% RH).

Citric acid and trisodium citrate were combined together in the following weight proportions: 16.51% and 83.49%, respectively. The mixture was blended at 47 rpm for 10 min using a Turbula T2F mixer.

The blend was micronised using an Atritor M3 fluid energy mill with a venturi pressure of 8 bar and grinding pressure of 2 bar. The blend was fed directly into the hopper at a target feed rate of ˜2 g/min. The produced powder (Component 4B) was collected into a single collection jar under reduced humidity (20% RH).

The micronised nitrite solid (component 4A) and the micronised citric acid solid (component 4B) were then blended in a ratio of 9:1 w/w citrate solid:nitrite solid, using a Turbula T2F mixer at 46 rpm for 20 minutes, resulting in the powder composition of Reference Example 4.

### NOx Evolution

Examples 1A, 2, 3 and 4 were loaded into an APTAR Unidose nasal spray (https://www.aptar.com/products/pharmaceutical/uds/), which was supported in a rig 30 cm above a petri dish (9.8 cm diameter) containing agarose with Hanks' balanced salt solution and a pH indicator (phenol red). FIG. 1 shows the deposition pattern of the powder by virtue of localised pH modification by the particles where they land.

Immediately after application the plate was transferred into a sealed chamber and the oxides of nitrogen (NOx) were measured by Selected Ion Flow Tube Mass

Spectrometry (SIFT-MS) over a period of 15 minutes. All powders, irrespective of their method of preparation, evolved nitric oxide. However, differences in the total quantity of NOx evolved are seen between the four powders over the course of fifteen minutes.

It should be noted that the agarose is buffered at neutral to slightly alkaline pH, which should inhibit the reaction, but the particles are able to overcome this buffering effect in the short term and counter-act the buffering in a localised area. The table below and FIG. 2 show the cumulative NO generation for Examples 1A, 2, 3 and 4. The cumulative NO/nmols per mg of nitrite normalises the results of the experiments for the % of nitrite in the powder.

| | Cumulative NO / | Cumulative NO / |
|---|---|---|
| Example | nmols | nmols · mgNitrite⁻¹ |
| EXAMPLE 1A | 2410 | 526 |
| EXAMPLE 3 | 1571 | 322 |
| EXAMPLE 4 | 562 | 120 |
| EXAMPLE 2 | 2393 | 552 |

### Coated Solid Powder Compositions

### Example 5: Particles Coated with Hydrophobic Materials DPPC or Mesoporous Silica

Example 1B was blended with mesoporous silica in a ratio of 1:1 w/w, using a Turbula T2F mixer at 46 rpm for 20 minutes, resulting in the powder composition of Example 5A.

Example 1B was blended with DPPC in a ratio of 1:1 w/w, using a Turbula T2F mixer at 46 rpm for 20 minutes, resulting in the powder composition of Example 5B.

Example 3 was blended with mesoporous silica in a ratio of 1:1 w/w, using a Turbula T2F mixer at 46 rpm for 20 minutes, resulting in the powder composition of Example 5C.

Example 3 was blended with DPPC in a ratio of 1:1 w/w, using a Turbula T2F mixer at 46 rpm for 20 minutes, resulting in the powder composition of Example 5D.

### Example 6: Particles Coated with PLGA

A PLGA RG 502 H solution was prepared by dissolving 1.5 g of PLGA to about 30 mL of DCM to form a clear and colourless solution. 1.5 g Example 1B was added to this solution with stirring to form a 1:1 w/w ratio feed suspension 6A as a visually uniform white suspension.

Similarly, a separate PLGA RG 502 H solution was prepared by dissolving 1.5 g of PLGA to about 30 mL of DCM to form a clear and colourless solution. 1.5 g Example 3 was added to this solution with stirring to form a 1:1 w/w ratio feed solution 6B as a visually uniform white suspension.

The feed suspensions were spray-dried using a Buchi B290 spray dryer according to the method detailed above. Spray drying parameters are summarised below.

| | | Outlet | Atomisation | Liquid |
|---|---|---|---|---|
| | Feed | Temperature | pressure | Feed rate |
| Example | suspension | (° C.) | (bar) | (g/min) |
| 6A | 6A | 28-29 | 1 | 2.0 |
| 6B | 6B | 29-30 | 1 | 2.0 |

In a reduced humidity environment (28% RH) sample vials were laid horizontally in individual weighing boats. The lids were removed and the openings were covered with foil with holes (pierced using a needle). Samples were transferred to an Edwards Super Modulyo freeze dryer set to 25° C. and vacuum dried for 24 h (maximum vacuum pressure observed was ˜0.1 mbar). Following vacuum drying, samples were transferred to a low humidity (˜24% RH) environment and overlaid with nitrogen. Vials were then sealed with Parafilm and sealed into foil pouches with desiccant for storage at 2-8° C.

Particle size distribution measurements were then taken using a Sympatec HELOS particle size analyser equipped with an R3 lens (0.5-175.0 μm range) and a ASPIROS dispersion unit. Dispersal was achieved using compressed air at a pressure of 3.00 bar and a depression of 60 bar. Measurements were made in triplicated.

The resultant particles size distribution measurements were as follows:

| | Mean Particle size (n = 3) by laser diffraction (μm) | | | |
|---|---|---|---|---|
| Example | X₁₀ | X₅₀ | X₉₀ | VMD |
| 6A | 0.92 | 4.17 | 11.07 | 5.23 |
| 6B | 0.89 | 3.50 | 7.58 | 4.05 |

VMD = volume mean diameter

### Example 7: NOx Evolution of Coated Particles

An aliquot of the powder sample (30 mg) was deposited in a 60 mm petri dish. Cellulose filter paper (50 mm diameter) was placed over the top of the sample, and light pressure applied. Sodium phosphate solution (10 mM, 250 μl) was dispensed onto the cellulose filter paper. The sample was immediately placed into a 650 ml chamber, which was sealed, and then humified air was pulled through the chamber at 650 ml/min for thirty minutes. The air stream from the outfeed was analysed by Single Ion Flow Tube Mass Spectrometry (SIFT-MS).

| | | Average | Average cumulative |
|---|---|---|---|
| | | cumulative | NO / nmols per mg |
| Example | Description | NO / nmols | sodium nitrite |
| EXAMPLE 7A | Spray-dried powder of sodium | 22141 | 6044 |
| | nitrite (1.5M) and citric acid | | |
| | (1M, pH 4.0) with vacuum | | |
| | drying (as in Example 1) | | |
| EXAMPLE 7B | Powder of Example 7A spray- | 4806 | 3342 |
| | dried with PLGA RP 502H | | |
| | (1:1 w/w ratio) as per | | |
| | Example 6 | | |
| EXAMPLE 7C | Powder of Example 7A spray- | 4973 | 4382 |
| | dried with PLGA RP 503H | | |
| | (1:1 w/w ratio) as per | | |
| | Example 6 | | |
| EXAMPLE 7D | Powder of Example 7A | 4061 | 3010 |
| | blended with magnesium | | |
| | stearate (1:1 w/w ratio) as per | | |
| | Example 5 | | |
| EXAMPLE 7E | Powder of Example 7A | 4088 | 2786 |
| | blended with magnesium | | |
| | stearate (1:1 w/w ratio) as per | | |
| | Example 5 | | |
| EXAMPLE 7F | Powder of Example 7A | 7341 | 3294 |
| | blended with magnesium | | |
| | stearate (1:1 w/w ratio) as per | | |
| | Example 5 | | |

### Biological Assessment of Solid Powder Compositions

### Example 8: Assessment of the Efficacy of Four Formulations Against Pseudomonas Aeruginosa

Petri dishes containing Nutrient Agar (NA, available from AcuMedia) were prepared and allowed to set. A Pseudomonas aeruginosa (ATCC 9027) inoculum was prepared in phosphate buffered saline (PBS, Sigma-Aldrich) and serially diluted to a final concentration of 1×10⁵ CFU mL⁻¹. 100 mL of inoculum was pipetted onto NA plates, spread, and allowed to dry at room temperature for 15 minutes. Lids were removed from the inoculated agar plates and the open plates were placed inside the Aptar Unidose nasal spray.

Aptar delivery devices containing either Example 1A, Example 3, Reference Example 4 or Example 2 powder were attached to the Aptar nasal spray devices and the powder was nebulized (approximately 50 mg dose) onto the agar plates. The table below shows the Examples used for each Formulation.

| | |
|---|---|
| Formulation 1 | Example 1A |
| Formulation 2 | Example 3 |
| Formulation 3 | Reference Example 4 |
| Formulation 4 | Example 2 |

After 5 seconds, the agar plate lids were replaced, and the agar plates were incubated for 16 hours at 37° C.±2° C. Following incubation, the plates were photographed. For all plates, three biopsy punches were taken from a 2×2 cm area in the centre of the agar plate. Sterile swabs moistened with PBS were used to remove the bacteria from each biopsy, any cells were suspended in 10 mL PBS before sonication for 5 minutes, serial dilution and were plated onto NA.

Negative control plates that were not exposed to nebulized powder, and positive control plates that had the addition of 1 mL bleach, were also tested concurrently. All testing was performed in quintuplicate.

For each test item, three replicates were randomly chosen, and DNA was extracted from 400 μL per biopsy using the DN easy Blood & tissue Kit (Qiagen), according to manufacturer's instructions. Samples were eluted in a final volume of 100 μL in AE buffer.

For each extraction, qPCR was performed in triplicate, using the QuantiNova Pathogen and IC kit (Qiagen) according to manufacturers instructions. Individual reaction tubes contained a final concentration of 16 μM for each primer and 5 μM labelled probes.

Cycle conditions were as follows: 50° C. for 10 min, 95° C. for 2 min, 35 cycles of 95° C. for 5 sec, 55° C. for 30 sec, 72° C. for 1 min. Each assay run was validated by positive (P. aeruginosa) and negative (RNase free water) controls. Data was analysed using the Q-Rex software (Qiagen) to obtain Cq values from a predetermined threshold value. For each sample, mean Cq values were compared to a standard curve with an established range of 1×10² to 1×10⁸ CFU mL⁻¹, to calculate final sample concentration in Log₁₀ CFUmL⁻¹.

TABLE 1
Average recoveries and reductions of Pseudomonas aeruginosa of
three biopsy punches taken at the centre of nutrient agar seeded with 1 ×
10⁵ CFU mL⁻¹, following treatment with formulation 1, 2, 3, 4
and bleach compared to an untreated negative control (N = 5).

| | Average | Average | |
|---|---|---|---|
| | recovery ± SD | reduction ± SD | |
| | (Log₁₀CFU | (Log₁₀CFU | Statistical |
| Test Agents | mL⁻¹) | mL⁻¹) | significance |
| Negative Control | 7.44 ± 0.17 | N/A | N/A |
| Formulation 1 | 0.00 ± 0.00 | 7.44 ± 0.00 | *** |
| Formulation 2 | 3.52 ± 3.12 | 3.91 ± 3.12 | * |
| Formulation 3 | 1.36 ± 2.13 | 6.08 ± 2.13 | ** |
| Formulation 4 | 0.00 ± 0.00 | 7.44 ± 0.00 | *** |
| Positive Control | 0.00 ± 0.00 | 7.44 ± 0.00 | *** |
| (bleach) | | | |

SD = standard deviation,
CFU = colony forming units,
N/A = not applicable,
* = p < 0.05,
** = p < 0.01,
*** = p < 0.001.

An average of P. aeruginosa recovery of 7.44±0.17 Log₁₀ CFU mL⁻¹ was observed from biopsies taken from the negative control plate. Average P. aeruginosa recoveries of 3.52±3.12 and 1.36±2.13 Log₁₀ CFU mL⁻¹ were observed from biopsies taken from Formulations 2 and 3. No viable P. aeruginosa was recovered from biopsies taken from Formulations 1 and 4 or the positive control plate.

TABLE 2
Molecular quantification of P. aeruginosa of biopsy
punches taken from nutrient agar seeded with 1 × 10⁵ CFU
mL⁻¹, following treatment with formulation 1, 2, 3,
4 and bleach compared to an untreated negative control.

| | Average Cq | Quantification ± SD | Statistical |
|---|---|---|---|
| Test Agents | Reading | (Log₁₀CFU mL⁻¹) | significance |
| Negative Control | 16.93 | 6.65 ± 0.18 | N/A |
| Formulation 1 | 33.91 | 1.15 ± 0.33^{#} | *** |
| Formulation 2 | 25.44 | 3.94 ± 1.85 | N/A |
| Formulation 3 | 25.59 | 2.73 ± 1.18 | ** |
| Formulation 4 | 31.27 | 1.78 ± 0.21^{#} | *** |
| Positive Control | 32.55 | 1.52^{~} | N/A |
| (bleach) | | | |

SD = standard deviation,
CFU = colony forming units.
^{#}= Quantification was below the limit of detection.
^{~}= The quantification of the positive control samples was performed to N = 1 so no standard deviation could be calculated.
N/A = not applicable,
** = p < 0.01,
*** = p < 0.001.

Significant reductions in the recovery of viable P. aeruginosa were observed from biopsies taken from the nutrient agar plates seeded with a 1×10⁵ CFU mL⁻¹ inoculum following treatment with Formulation 1 and Formulation 4. Powders when compared to the untreated negative control, as no viable P. aeruginosa were recovered. Molecular quantification reflects the recovery from colony counts.

### Example 9: Effect of Powdered Compositions on the Sprouting of Human Umbilical Vein Endothelial Cells (HUVEC) in Spheroid-Based Cellular Angiogenesis Assay

10× concentrated stock solutions/suspensions of Examples 1B and 6A were prepared in basal medium (without supplement and FCS) by vortexing and pipetting. Subsequently, semi-log dilution series were prepared in the same medium.

| | Example 1B | Example 6A |
|---|---|---|
| # | [mg/mL] | [mg/mL] |
| 1 | 5.000 | 10.000 |
| 2 | 1.500 | 3.000 |
| 3 | 0.500 | 1.000 |
| 4 | 0.150 | 0.300 |
| 5 | 0.050 | 0.100 |
| 6 | 0.015 | 0.030 |
| 7 | 0.005 | 0.010 |

### Endothelial Cells

Cells: HUVEC, primary human umbilical vein endothelial cells (PromoCell, Heidelberg, Germany), passage 3 to 4.
Morphology: adherent, cobblestone-like growing as monolayer
Medium: endothelial cell growth and basal medium (ECGM/ECBM, PromoCell)
Subculture: split 1:3; every 3-5 days, seed out at ca. 1×10⁴ cells/cm²
Incubation: at 37° C. with 5% CO₂
Doubling Time: 24-48 hours
Storage: frozen with 70% medium, 20% FCS, 10% DMSO at about 1×10⁶ cells/ampoule
Origin: human umbilical vein, pooled donors

### Test Method

The experiments were pursued in modification of the originally published protocol (Korff and Augustin: J Cell Sci 112:3249-58, 1999). In brief, spheroids were prepared as described (Korff and Augustin: J Cell Biol 143:1341-52, 1998) by pipetting 400 HUVEC in a hanging drop on plastic dishes to allow overnight spheroid aggregation. 50 HUVEC spheroids were then seeded in 0.9 ml of a collagen gel and pipetted into individual wells of a 24 well plate to allow polymerization. Preincubated test samples were added after 30 min by pipetting 100 μl of a 10-fold concentrated working solution on top of the polymerized gel (final assay concentrations see table 1). Plates were incubated at 37° C. for 24 hours and fixed by adding 4% PFA (Roth, Karlsruhe, Germany).

### Quantification

Sprouting intensity of HUVEC spheroids treated with the test samples were quantitated by an image analysis system determining the cumulative sprout length per spheroid (CSL). Pictures of single spheroids were taken using an inverted microscope and the digital imaging software NIS-Elements BR 3.0 (Nikon). Subsequently, the spheroid pictures were uploaded to the homepage of the company Wimasis for image analysis. The cumulative sprout length of each spheroid was determined using the imaging analysis tool WimSprout. The mean of the cumulative sprout length of 10 randomly selected spheroids was analyzed as an individual data point. Mean and SD values of each triplicate were converted into % of basal control.

### Result

FIG. 3 shows the CSL relative to the basal control of Examples 1B and 6A. The effect of Example 1B (spray-dried particles with no coating) is small compared to the basal control. In contrast, the PLGA-coated particles of Example 6A show a significant dose-dependent effect compared with the basal control. This indicates that the coated particles provide a localized environment that capable of the acidification of nitrite despite being in a substantially neutral environment.

FIG. 4 shows a schematic of a wound dressing 100 of the present invention having a backing layer 102, a nitric oxide generating layer 106 and a removable protective layer 108. The wound dressing 100 may be in a hermetically sealed packaging prior to use. When the wound dressing 100 is required, the wound dressing 100 may be removed from any packaging. The removable protective layer 108 may be removed from the wound dressing 100 to expose the nitric oxide generating layer 106. The wound dressing may be applied to a wound of a subject by placing the nitric oxide generating layer 106 onto the subject's wound. Water may optionally be added to the exposed nitric oxide generating layer 106 prior to application of the wound dressing.

Adhesive sections 104 may adhere to the subject to adhere the wound dressing to the subject. The adhesive sections and the backing layer 102 may help to seal the wound dressing to the subject. FIG. 4 is not to scale.

### Example 10: NO Release from a Carboxymethylcellulose (CMC) One-Part Wound Dressing Containing a Nitrite Source and a Proton Source (FIG. 5)

A carboxymethylcellulose based fabric (120 g/m², SFM Ltd, Mercury) was impregnated with a powder (20 g/m²) containing a nitrite source and proton source similar to Example 1A, using high intensity alternating electric fields.

Nitric oxide release was measured by applying the one-part dressing to a laboratory wound model and analysing evolved gaseous nitric oxide by Selected-Ion Flow Tube Mass Spectrometry (SIFT-MS). A schematic of the apparatus used is shown in FIG. 7.

A laboratory wound model consisting of a shallow 5 cm diameter cylindrical cup with an infeed and an outfeed, set within a heated stainless-steel plate (30° C.) at an incline angle of 2° towards the infeed, was used. Within the cup were placed two 4.9 cm diameter cellulosic filter papers pre-saturated with sodium chloride solution (0.9% w/v), making a surrogate “wound bed.”

The infeed was connected to a syringe pump that imbibed sodium chloride solution (0.9% w/v) at a rate of 0.4 ml/hr for the duration of the test. A 5×5 cm square piece of one-part dressing was cut from the main sample and weighed.

The dressing was placed onto the “wound bed.” For leak prevention in the test the sample was surrounded with a frame of blank carboxymethylcellulose based fabric (120 g/m², SFM Ltd, Mercury) 10×10 cm, with circa 5×5 cm window cut from the centre.

A stainless-steel container 10×10 cm, with a mesh grid across the surface, was placed on top of the dressing with the mesh grid facing down and touching the dressing to act as a weight.

A measurement chamber (1100 ml plastic box) with an infeed and an outfeed to the SIFT-MS was placed onto the stainless-steel plate of the laboratory wound model, which covered the dressing, surrounding fabric frame and stainless-steel container in their entirety. The plastic box was weighed down with a circa 1 kg weight.

To the outfeed of the plastic box was connected tubing, which connected to a Dreschel bottle containing 2 mM sodium hydroxide solution, followed by a Dreschel containing silica beads and finally to the inlet of the SIFT-MS. Air was drawn through the system at approximately 30 ml/min and analysed by SIFT-MS. The NO release profile for 2000 minutes is shown in FIG. 5.

### Example 11: NO Release from a Multi-Layer Composite Dressing Containing a One-Part Nitric Oxide Generating Layer Containing a Nitrite Source and a Proton Source (FIG. 6)

A hydroactive wound pad made from a polyester fibre with super absorbing powder (136 g/m² base weight, Freudenberg, M1520) was impregnated with a powder (20 g/m²) containing a nitrite source and proton source similar to Example 1A, using high intensity alternating electric fields.

Nitric oxide release was measured by applying the one-part dressing to a laboratory wound model and analysing evolved gaseous nitric oxide by Selected-Ion Flow Tube Mass Spectrometry (SIFT-MS).

A laboratory wound model consisting of a shallow 5 cm diameter cylindrical cup with an infeed and an outfeed, set within a heated stainless-steel plate (30° C.) at an incline angle of 2° towards the infeed, was used. Within the cup were placed two 4.9 cm diameter cellulosic filter papers pre-saturated with sodium chloride solution (0.9% w/v), making a surrogate “wound bed.”

The infeed was connected to a syringe pump that imbibed sodium chloride solution (0.9% w/v) at a rate of 0.4 ml/hr for the duration of the test. A 5×5 cm square piece of one-part dressing was cut from the main sample and weighed. To the wound contact surface of this sample was laminated a 5×5 cm square piece of absorbent wicking material (20 g/m² polypropylene, Daltex® Absorb, Don & Low Ltd).

The laminate dressing was placed onto the “wound bed.” For leak prevention in the test the sample was surrounded with a frame of blank carboxymethylcellulose based fabric (120 g/m², SFM Ltd, Mercury) 10×10 cm, with circa 5×5 cm window cut from the centre. A stainless-steel container 10×10 cm, with a mesh grid across the surface, was placed on top of the dressing with the mesh grid facing down and touching the dressing to act as a weight.

A measurement chamber (1100 ml plastic box) with an infeed and an outfeed to the SIFT-MS was placed onto the stainless-steel plate of the laboratory wound model, which covered the dressing, surrounding fabric frame and stainless-steel container in their entirety.

The plastic box was weighed down with a circa 1 kg weight. To the outfeed of the plastic box was connected tubing, which connected to a Dreschel bottle containing 2 mM sodium hydroxide solution, followed by a Dreschel containing silica beads and finally to the inlet of the SIFT-MS. Air was drawn through the system at approximately 30 ml/min and analysed by SIFT-MS. The NO release profile for 2000 minutes is shown in FIG. 6.

1. A wound dressing for treating a wound, the wound dressing comprising a nitric oxide generating layer for generating nitric oxide by the acidification of a nitrite salt, wherein the nitric oxide generating layer includes a solid powder nitrite salt component and a solid proton source component, wherein the solid powder nitrite salt component and the solid powder proton source component are provided by:
a. a blend of one or more individual particles containing a nitrite salt and one or more individual particles containing a proton source;
b. one or more individual particles that each contain a nitrite salt and a proton source;
c. an agglomeration of particles, wherein the agglomeration of particles includes one or more individual particles containing a nitrite salt, one or more individual particles containing a proton source and optionally a binding agent;
d. an agglomeration of particles, wherein the agglomeration of particles includes one or more individual particles that contain a nitrite salt and a proton source and optionally a binding agent; or
e. combinations thereof.

2. The wound dressing of claim 1 wherein the nitric oxide generating layer includes a dry wound dressing substrate.

3. The wound dressing of claim 2 wherein the dry proton source component includes a solid powder proton source component, and the solid powder proton source component is admixed with the dry wound dressing substrate.

4. The wound dressing of claim 2 wherein at least part of the dry proton source component forms part of the dry wound dressing substrate.

5. The wound dressing of claim 2 wherein the dry proton source component includes a solid powder proton source component and further part of the dry proton source component forms part of the dry wound dressing substrate.

6. The wound dressing of claim 2 wherein the dry wound dressing substrate is made up of woven or non-woven fibres.

7. The wound dressing of claim 1 wherein all of the components of the nitric generating layer are dry components.

8. The wound dressing of claim 1 wherein the water content of the nitric oxide generating layer is 10% or less, 5% or less, 2% or less or 1% or less based on the weight of the nitric oxide generating layer.

9. The wound dressing of claim 1 wherein the wound dressing is a one-part wound dressing.

10. The wound dressing of claim 1 wherein the wound dressing includes one or more further layers in addition to the nitric oxide generating layer.

11. The wound dressing of claim 1, wherein one or more of the individual particles or agglomeration of particles are blended with or coated with an excipient for affecting the rate of water ingress into particles and/or an excipient for affecting the kinetics of the formation of nitric oxide from the particles.

12. The wound dressing of claim 11 wherein the excipient for affecting the rate of water ingress into particles is a polyols or a hydrophobic material, such as a phospholipid, magnesium stearate or colloidal silica, and/or the excipient for affecting the rate of water ingress into particles is a nitric oxide or nitric oxide precursor sequestering material, such as thiols, alcohols, amines or amides.

13. The wound dressing of claim 11 wherein the particles containing a nitrite salt and a proton source are formed by spray-drying a mixture containing a nitrite salt solution and a proton source solution.

14. The wound dressing of claim 11, wherein the blend of one or more individual particles containing a nitrite salt and one or more individual particles containing a proton source is formed by either (a) micronizing a nitrite salt solid with a proton source solid; or (b) combining two solids by:
(i) spray-drying or lyophilising a solution containing a nitrite salt,
(ii) spray-drying or lyophilising a solution containing a proton source, and
(iii) blending the solids generated in steps (i) and (ii).

15. The wound dressing of claim 1 wherein the proton source comprises an acid precursor, such as an ester or a photoacid.

16. The wound dressing of claim 1 wherein the wound dressing includes one or more further dry layers adjacent to nitric oxide generating layer.

17. The wound dressing according to claim 1 further comprising one or more further layers adjacent to the nitric oxide generating layer provided that the water content of any layer adjacent to the nitric oxide generating layer is 5% or less, 2% or less or 1% or less based on the weight of the layer adjacent to the nitric oxide generating layer.

18. The wound dressing of claim 1 wherein the wound dressing includes an anti-microbial agent.

19. The wound dressing of claim 1, further comprising a low moisture permeability packaging.

20. The packaged wound dressing of claim 19 wherein the low moisture permeability packaging includes one or more low moisture permeability materials in the walls of the packaging.

21. (canceled)

22. (canceled)

23. (canceled)

24. (canceled)

25. (canceled)
